(19) United States
(12) Patent Application Publication
(10) Pub. No.: US 2022/0296521 A1
(43) Pub. Date: Sep. 22, 2022

(54) CARRIER-BASED FORMULATIONS AND RELATED METHODS

(71) Applicant: Respira Therapeutics, Inc., Albuquerque, NM (US)

(72) Inventors: Danforth P. Miller, San Carlos, CA (US); Thomas E. Tarara, Burlingame, CA (US); Jeffry G. Weers, Half Moon Bay, CA (US)

(73) Assignee: Respira Therapeutics, Inc., Albuquerque, NM (US)

(21) Appl. No.: 17/617,902

(22) PCT Filed: Jun. 10, 2020
(86) PCT No.: PCT/US2020/036944

§ 371 Date: Dec. 9, 2021

Related U.S. Application Data
(60) Provisional application No. 62/859,423, filed on Jun. 10, 2019.

(51) Int. Cl. A61K 9/16; A61K 31/58; A61K 9/00; A61K 31/569; A61P 11/00

(52) U.S. Cl. CPC A61K 9/1617; A61K 9/1694; A61K 31/58; A61K 9/0075; A61K 31/569; A61P 11/00

(57) ABSTRACT

Provided herein are carrier-based dry powder formulations to be administered as dry powders for inhalation and that enable improved targeting within the respiratory tract (e.g., to the lower respiratory tract) of patients. The carrier-based dry powder formulations described herein have a desired size and impaction parameter that promotes targeted delivery of formulations to regions of the lungs and reduce the loss of drugs in the formulation to deposition in other regions of the respiratory tract (e.g., URT). Also provided herein are methods of producing the formulations, methods of making the formulations, and methods of aerosolizing and using the formulations to treat disease.

### CROSS-REFERENCE TO RELATED APPLICATION

This application claims the benefit of and priority to U.S. Provisional Application No. 62/859,423, filed Jun. 10, 2019, which is incorporated herein by reference in its entirety.

### FIELD

The present disclosure is related to carrier-based dry powder formulations and methods of preparing and using such formulations. More particularly, the present disclosure relates to carrier-based dry powder formulations for improved delivery to the lungs and in particular the small airways, methods for preparing such formulations, and methods of using such formulations.

### BACKGROUND

The respiratory tract is divided into two principal regions, the upper respiratory tract (URT) comprising the mouth, larynx, and pharynx, and the lower respiratory tract (LRT) comprising the trachea and lungs. The respiratory tract may also be subdivided into the conducting zone (nose, pharynx, larynx, trachea, bronchi, bronchioles, and terminal bronchioles) where air breathed in is filtered, warmed, and moistened, and the respiratory zone (respiratory bronchioles, alveolar ducts, alveoli) where gas exchange occurs. Within the lungs, the conducting zone encompasses the first 16 generations and the respiratory zone encompasses generations 17-23.

Unwanted deposition of particles in the URT may lead to adverse events in the mouth and throat (e.g., opportunistic infections, dysphonia), and systemic circuit. In order for particles to be deposited in the lung periphery, the particles must first bypass inertial impaction in the URT and large airways, after which they must sediment in the small airways or alveoli before being exhaled. The Stokes number (Stk) defines the probability that a particle will diverge from the streamlines of a carrier gas and deposit by inertial impaction in the respiratory tract, which is given in Eq. 1:

$$S t k = \frac{{ρ}_{p} {d}_{p}^{2} u}{18 μ D} ∼ \frac{{d}_{a}^{2} Q}{18 μ D} \qquad ( 1 )$$

where dₚ, ρₚ, and dₐ are the particle diameter, density, and aerodynamic diameter, respectively, μ and μ are the linear velocity and dynamic viscosity of the carrier gas, and D is a characteristic length scale equal to the diameter of the airspace. The volumetric flow rate, Q, is often used to approximate the linear velocity. The product dₐ²Q is termed the “impaction parameter.” The larger the impaction parameter, the more likely particles will deposit by inertial impaction and not reach the lung periphery.

Particles that are not captured by inertial impaction may settle in the respiratory tract under the action of gravity by a process termed “gravitational sedimentation.” The terminal settling velocity for a spherical particle, v, is given by Eq. 2:

$$v = \frac{{ρ}_{p} {d}_{p}^{2}}{18 μ} g ∼ \frac{{d}_{a}^{2}}{18 μ} g \qquad ( 2 )$$

where g is the acceleration due to gravity. The probability that a particle will deposit by gravitational sedimentation increases with the square of the aerodynamic diameter of the particle, and with increasing residence time in the airways.

Current marketed dry powder inhalers for the treatment of asthma and COPD are comprised of either adhesive mixtures of coarse lactose carrier particles and micronized drug particles (lactose blends, LB), or coarse spheronized agglomerates of micronized drug particles (SPH). These two formulation technologies have one thing in common: micronized drug particles that remain adhered to the carrier or in the spheronized agglomerates of particles following emission from a dry powder inhaler will be deposited in the URT.

For lactose blends, the adhesive forces between drug and carrier must be strong enough to maintain the adhesive mixture through the powder filling process and in storage over its shelf-life (i.e., no segregation between the fine micronized drug particles and coarse carrier particles within the receptacle), yet weak enough to enable dispersion of drug from carrier during aerosolization from a dry powder inhaler. Unfortunately, dispersion of drug in these formulations is poor with 50-90% of the emitted dose lost in the URT. Inertial impaction also contributes to significant drug deposition in the large airways, and only 5% to 15% of the drug makes its way to the peripheral regions of the lungs.

An empirical relationship between the impaction parameter and deposition in the URT of adult humans was established by Stahlhofen et al. (J Aerosol Med. 1989; 2:285-308) for monodisperse aerosols. The experimental data for URT deposition of monodisperse aerosols as a function of impaction parameter are plotted in FIG. 1, and the empirical fit to the data is given by Eq. 3:

URT Deposition=1−(4.17×10⁻⁶(dₐ²Q)^{1.7}+1)⁻¹ (3)

As expected, increases in dₐ²Q lead to corresponding increases in URT deposition. The shaded area in FIG. 1 represents the range of dₐ²Q values that result in the 50-90% mean deposition in the URT observed for current marketed products comprising spheronized agglomerates of micronized drug particles (SPH) and lactose blend (LB) formulations (dₐ²Q=1452 to 5286 μm² L min⁻¹). These types of formulations exhibit bimodal particle size distributions, with the fine mode comprising free micronized drug, and the coarse mode comprising agglomerated drug particles in SPH, or drug adhered to coarse carrier particles in LB. Within the shaded region of FIG. 1, URT deposition varies from about 5% to 95%, with maximal variability in URT deposition for dₐ²Q values between about 1500 μm² L min⁻¹ to 3000 μm² L min⁻¹. Unfortunately, this region is where the impaction parameters of marketed dry powder products comprising LB and SPH particles fall. Newman (Exp Opin Drug Deliv. 2014; 11:365-378) opined that:
“A patient can adhere fully to the treatment regimen but gets no benefit because the inhaler is not used correctly. Conversely, the patient may have perfect inhaler technique, but gets no benefit because the inhaler is not used often enough.”

The influence of the impaction parameter on regional deposition of monodisperse liquid droplets containing albuterol was assessed with gamma scintigraphy (Usmani et al. Am J Respir

Crit Care Med. 2005; 172:1497-1504). As shown in FIG. 2, URT deposition increases with increasing dₐ²Q. Significant increases in peripheral lung delivery (labelled as P+EXH), including the small airways, is observed for dₐ²Q values less than 500 μm² L min⁻¹.

Unfortunately, conventional dry powder formulations comprising adhesive mixtures of carrier and micronized drug are not able to achieve mean dₐ²Q values less than 500 μm² L min⁻¹, much less the dₐ²Q values needed to largely bypass URT deposition (i.e., dₐ²Q˜100 μm² L min⁻¹). This disclosure is directed to dry powder formulations and methods of preparing said formulations that achieve target values of the impaction parameter for effective delivery of dry powder formulations to the LRT, and in particular into the small airways.

### BRIEF SUMMARY

Provided herein are formulations and methods for delivering formulations comprising pharmaceutical compositions to the airways of the lungs.

In some embodiments, a carrier-based dry powder formulation is provided that includes a plurality of drug particles adhered to carrier particles forming particle agglomerates having a mass median impaction parameter (MMIP) value between 50 and 2500 μm² L min⁻¹.

In some embodiments, a carrier-based dry powder formulation is provided that includes a plurality of drug particles adhered to fine carrier particles forming particle agglomerates having a mass median impaction parameter (MMIP) value between 500 and 2500 μm² L min⁻¹.

In some embodiments, a carrier-based dry powder formulation is provided that includes a plurality of drug particles adhered to extrafine carrier particles forming particle agglomerates having a mass median impaction parameter (MMIP) value between 50 and 500 μm² L min⁻¹.

In some embodiments, a carrier-based dry powder formulation is provided that includes a plurality of drug particles adhered to extrafine leucine carrier particles forming particle agglomerates having a mass median impaction parameter (MMIP) value between 50 and 500 μm² L min⁻¹.

In some embodiments, a carrier-based dry powder formulation is provided that includes a plurality of drug particles adhered to fine leucine carrier particles forming particle agglomerates having a mass median impaction parameter (MMIP) value between 500 and 2500 μm² L min⁻¹.

In some embodiments, a method of preparing a carrier-based dry powder formulation is provided. In some embodiments, the method includes: preparing carrier particles comprising a median aerodynamic diameter (Dₐ) less than 3 μm; adding a non-solvent to the carrier particles to form a suspension; preparing a drug solution comprising a drug and a solvent that is miscible with the non-solvent; adding the drug solution to the suspension of carrier particles in the non-solvent while mixing to precipitate the drug particles and thereby forming a co-suspension of drug particles and carrier particles in the non-solvent; and removing the non-solvent to form a dry powder comprising an adhesive mixture of drug particles adhered to the carrier particles, wherein the adhesive mixture has a mass median impaction parameter (MMIP) value between 50 and 2500 μm² L min⁻¹.

In some embodiments, a method of preparing a carrier-based dry powder formulation is provided, the method including the steps of: preparing an aqueous solution comprising leucine;

drying the aqueous solution to produce fine leucine carrier particles comprising a median aerodynamic diameter (Dₐ) from 1 μm to 3 μm; adding a non-solvent to the fine leucine carrier particles to form a suspension; preparing a drug solution comprising a drug and a solvent that is miscible with the non-solvent; adding the drug solution to the suspension of fine leucine carrier particles in the non-solvent while mixing to precipitate the drug particles and thereby forming a co-suspension of drug particles and fine leucine carrier particles in the non-solvent; and removing the non-solvent to form a dry powder comprising an adhesive mixture of drug particles adhered to the fine leucine carrier particles, wherein the adhesive mixture has a mass median impaction parameter (MMIP) value between 500 and 2500 μm² L min⁻¹.

In some embodiments, a method of preparing a carrier-based dry powder formulation is provided, the method including the steps of: preparing an aqueous solution comprising leucine; drying the aqueous solution to produce extrafine leucine carrier particles comprising a median aerodynamic diameter (Dₐ) less than 1000 nm; adding a non-solvent to the extrafine leucine carrier particles to form a suspension; preparing a drug solution comprising a drug and a solvent that is miscible with the non-solvent; adding the drug solution to the suspension of extrafine leucine carrier particles in the non-solvent while mixing to precipitate the drug particles and thereby forming a co-suspension of drug particles and extrafine leucine carrier particles in the non-solvent; and removing the non-solvent to form a dry powder comprising an adhesive mixture of drug particles adhered to the extrafine leucine carrier particles, wherein the adhesive mixture has a mass median impaction parameter (MMIP) value between 50 and 500 μm² L min⁻¹.

In some embodiments, a method of treating a disease in a subject is provided. In some embodiments, the method comprises administering to a subject in need thereof an effective amount of a carrier-based dry powder formulation described herein, wherein the carrier-based dry powder formulation is administered to the subject via inhalation.

### BRIEF DESCRIPTION OF THE DRAWINGS

FIG. 1 shows a graph of URT deposition as a function of impaction parameter (Stahlhofen et al., J Aerosol Med. 1989; 2:285-308).

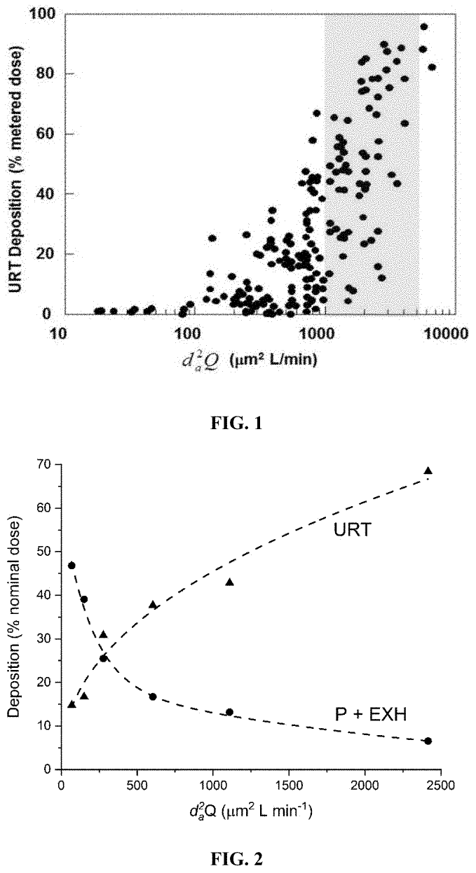

FIG. 2 shows the influence of the impaction parameter on particle deposition in the URT and lung periphery for monodisperse albuterol aerosols in adult asthmatics (adapted from Usmani et al., Am J Respir Crit Care Med. 2005; 172:1497-1504). dₐ=1.5, 3, 6 μm; Q=30, 60 L/min. Line sloping up is URT. Line sloping down is P+EXH.

FIG. 3 shows the aerodynamic diameter and volumetric flow rates required to achieve a target impaction parameter and URT deposition in adult subjects.

FIG. 4 shows X-ray powder patterns of precipitated ciclesonide and unprocessed starting material according to aspects of this disclosure.

FIG. 5 shows an overlay of X-ray powder diffraction patterns of powders comprising 1, 5, 10, and 20% w/w ciclesonide according to aspects of this disclosure.

FIG. 6 shows an overlay of X-ray powder diffraction patterns of ciclesonide drug substance, leucine carrier particles, and a 5% ciclesonide/leucine blend before and after exposure to elevated relative humidity (75% RH) according to aspects of this disclosure.

FIG. 7 shows a graph of assay and blend uniformity (% RSD of assay) of ciclesonide/leucine blends according to aspects of this disclosure.

FIG. 8 shows an overlay of X-ray powder diffraction patterns of powders comprising 1% and 5% fluticasone propionate according to aspects of this disclosure.

FIG. 9 shows flow rate dependence of lung dose measured using an Idealized Child Throat (ICT) or an Alberta Idealized (adult) Throat (AIT) according to aspects of this disclosure. All measurements were taken at ambient laboratory conditions (e.g., ˜20° C./40% RH) except for the datum measured at elevated RH (25° C./75% RH) using the ICT.

FIG. 10 shows a graph of moisture sorption isotherm of 1% ciclesonide/leucine blend and a benchmark hydrophobic carrier, DSPC:CaCl₂ according to aspects of this disclosure. Both isotherms were measured at 25° C.

FIG. 11 shows a plot of lung targeting (i.e., the ratio of TLD/URT deposition) for various ICS-containing formulations including CPI according to aspects of this disclosure.

FIG. 12 provides a table showing deposition in the device, pediatric throat, and lungs for three ICS formulations in the ICT model according to aspects of this disclosure.

FIGS. 13A-13F show graphs of aerodynamic particle size distributions (aPSD) generated with a NEXT GENERATION IMPACTOR™ (Copley Scientific, Shoreview, Minn.) for various ICS formulations according to aspects of this disclosure.

### DEFINITIONS

Throughout this disclosure and in the claims that follow, unless the context requires otherwise, the words “carrier-based dry powder formulation,” “carrier-based dry powder composition,” “carrier-based formulation,” and “carrier-based composition” are used interchangeably.

“Active ingredient”, “therapeutically active ingredient”, “active agent”, “drug” or “drug substance” as used herein means the active ingredient of a pharmaceutical, also known as an active pharmaceutical ingredient (API).

“Fixed dose combination” as used herein refers to a pharmaceutical product that contains two or more active ingredients that are formulated together in a single dosage form available in certain fixed doses.

“Carrier-free” formulations as used herein refer to composite particle formulations where the drug and excipients are present in the same particle.

“Carrier-based” formulations as used herein are comprised of interactive mixtures of drug particles adhered to carrier particles.

The term “fine” when referring to carrier particles described herein refers to particles having a geometric diameter between 2.5 μm and 5 μm. The fine carrier particles have median aerodynamic diameters for the primary carrier particles (Dₐ) between 1.0 μm and 3.0 μm.

The term “extrafine” when referring to carrier particles described herein refers to particles having a geometric diameter between 0.5 μm and 2.5 μm. The extrafine carrier particles have median aerodynamic diameters for the primary carrier particles (Dₐ) between 100 nm and 1000 nm.

“Amorphous” as used herein refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid-like properties occurs at a “glass transition”, typically defined as a second-order phase transition.

“Crystalline” as used herein refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically a first-order phase transition (“melting point”). In the context of the present invention, a crystalline active ingredient means an active ingredient with crystallinity of greater than 85%. In certain embodiments the crystallinity is suitably greater than 90%. In other embodiments, the crystallinity is greater than 95%. In other embodiments, the crystallinity is less than 10%, or less than 5%.

“Drug Loading” as used herein refers to the percentage of active ingredient(s) on a mass basis in the total mass of the formulation.

“Impaction Parameter” as used herein refers to the product of the aerodynamic diameter squared times the volumetric flow rate, i.e., dₐ²Q.

“Mass median diameter” or “MMD” or “x₅₀” as used herein means the median diameter of a plurality of particles, typically in a polydisperse particle population, i.e., consisting of a range of particle sizes. The X₅₀ values as reported herein are determined by laser diffraction (Sympatec Helos, Clausthal-Zellerfeld, Germany), unless the context indicates otherwise.

The term “geometric diameter” or “dᵣ” refers to the geometric diameter for a single particle. As used herein, the geometric diameter is the physical geometric size of a particle. The x₅₀, as described, represents the median geometric diameter of an ensemble of particles. The “aerodynamic diameter” of a particle, “dₐ”, is equal to the geometric diameter multiplied by the square root of the particle density.

“Tapped densities” or ρ_{tapped} as used herein were measured in a fashion similar to Method I, as described in USP <616> Bulk Density and Tapped Density of Powders. Tapped densities represent a closer approximation to particle density than poured bulk densities, with measured values that are approximately 20% less than the actual particle density.

“Median aerodynamic diameter of the primary particles” or Dₐ as used herein, is calculated from the mass median diameter of the bulk powder as determined via laser diffraction (x₅₀) at a dispersing pressure sufficient to create primary particles (e.g., 4 bar), and their tapped density, namely: Dₐ=x₅₀\sqrt{ρ_{tapped}}. In this disclosure, the term “median aerodynamic diameter of the carrier particles” is used interchangeably with “median aerodynamic diameter of the primary particles” and has the same definition.

“Mass median aerodynamic diameter” or “MMAD” as used herein refers to the median aerodynamic size of a plurality of particles, typically in a polydisperse population. The “aerodynamic diameter” is the diameter of a unit density sphere having the same settling velocity, generally in air, as a powder and is therefore a useful way to characterize an aerosolized powder or other dispersed particle or particle formulation in terms of its settling behavior. The aerodynamic particle size distributions (aPSD) and MMAD are determined herein by cascade impaction, using a NEXT GENERATION IMPACTOR™ (Copley Scientific). In general, if the particles are aerodynamically too large, fewer particles will reach specific regions of the lungs. If the particles are too small, a larger percentage of the particles may be exhaled. In contrast, dₐ represents the aerodynamic diameter of a single particle.

“Mass median impaction parameter” or “MMIP” as used herein refers to the mass median impaction parameter for a plurality of particles, typically in a polydisperse population. The MMIP utilizes the impaction parameter cutoffs for the stages in a NEXT GENERATION IMPACTOR™ as opposed to the size cutoffs.

“Nominal Dose” or “ND” as used herein refers to the mass of drug loaded into a receptacle (e.g., capsule or blister) in a non-reservoir based dry powder inhaler. ND is also sometimes referred to as the metered dose.

“Emitted Dose” or “ED” as used herein refers to an indication of the delivery of dry powder from an inhaler device after an actuation or dispersion event from a powder unit. ED is defined as the ratio of the dose delivered by an inhaler device to the nominal or metered dose. The ED is an experimentally determined parameter and may be determined using an in vitro device set-up which mimics patient dosing. ED is also sometimes referred to as the delivered dose (DD).

“Total Lung Dose” (TLD) as used herein, refers to the percentage of active ingredient(s) which is not deposited in an Alberta Idealized Throat (AIT) or an Idealized Child Throat (ICT), and instead is captured on a filter post-throat, following delivery of powder from a dry powder inhaler. The AIT represents an idealized version of the upper respiratory tract for an average adult subject. The ICT represents an idealized version of the upper respiratory tract for an average child (age 6 to 14). Data can be expressed as a percentage of the nominal dose or the emitted dose. Information on the AIT and ICT and a detailed description of the experimental setup can be found at: www.copleyscientific.com. The AIT models and experimental setup are described in more detail in Finlay, W H, and A R Martin, “Recent advances in predictive understanding respiratory tract deposition”, Journal of Aerosol Medicine, Vol. 21:189-205 (2008). The ICT models and experimental setup are described in more detail in the following: Golshahi, L, M L Noga, and W H Finlay, “Deposition of inhaled micrometer-sized particles in oropharyngeal airway replicas of children at constant flow rates”, Journal of Aerosol Science, Vol. 49:21-31 (2012); Golshahi, L, M L Noga, R B Thompson and W H Finlay, “In vitro deposition measurement of inhaled micrometer-sized particles in extrathoracic airways of children and adolescents during nose breathing”, Journal of Aerosol Science, Vol. 42:474-488 (2011); and Golshahi, L, R Vehring, M L Noga and W H Finlay, “In vitro deposition of micrometer-sized particles in extrathoracic airways of children during tidal oral breathing”, Journal of Aerosol Science, Vol. 57:14-21 (2013). The TLD can also be determined in vivo using techniques such as gamma scintigraphy or PET. Good correlations have been established between measurements conducted with in vitro throat models and in vivo deposition measurements.

“Fine particle fraction” (FPF) as used herein, refers to the percentage of active ingredient in the emitted dose with an aerodynamic size less than 5 μm. The aerodynamic particle size distributions (aPSD) is determined herein by cascade impaction, using a NEXT GENERATION IMPACTOR™. Fine particle fractions based on stage groupings (i.e., impaction parameters) are often reported. For example, the FPF_{S5-F} (i.e., the stage grouping from stage 5 to filter) represents particles with a dₐ²Q<165 μm² L min⁻¹.

“Humidity Index” as used herein refers to the ratio of the fine particle dose at 75% relative humidity (RH) to that at about 40% RH.

“Impaction parameter” as used herein refers to the parameter which characterizes inertial impaction in the upper respiratory tract. The parameter was derived from Stokes' Law and is equal to dₐ²Q, where dₐ is the aerodynamic diameter, and Q is the volumetric flow rate.

“Solids Content” as used herein refers to the concentration of active ingredient(s) and excipients dissolved or dispersed in the liquid solution or dispersion to be spray-dried.

“Primary particles” or “primary carrier particles” refer to the smallest divisible particles that are present in an agglomerated bulk powder. The primary particle size distribution is determined via dispersion of the bulk powder at high pressure and measurement of the primary particle size distribution via laser diffraction. A plot of size as a function of increasing dispersion pressure is made until a constant size is achieved. The particle size distribution measured at this pressure represents that of the primary particles.

“Q index” provides a measure of the flow rate dependence of pharmaceutical aerosols. The impactor version of the Q index utilizes the normalized differences in stage grouping (e.g., FPD_{S4-F}) between pressure drops of 1 kPa and 6 kPa, as opposed to size cutoffs. Drug products with a Q index greater than 40% are deemed to have a high flow rate dependence, those with 15% <Q index<40%, medium flow rate dependence, and those <15%, low flow rate dependence (see Weers and Clark. Pharm Res. 2017; 34:507-528). Alternatively, the Q index can be determined in vitro using the AIT or ICT throat models.

The term “about” refers to variations in numerical values typically encountered by one of skill in the art of respirable formulations, including variations of plus or minus 0.1%, 0.5%, 1%, 2%, 3%, 4%, 5%, 6%, 7%, 8%, 9%, or 10% of a numerical value described herein.

Throughout this specification and in the claims that follow, unless the context requires otherwise, the word “comprise”, or variations such as “comprises” or “comprising”, should be understood to imply the inclusion of a stated integer or step or group of integers or steps but not the exclusion of any other integer or step or group of integers or steps.

Unless otherwise stated, or clear from the context, numerical ranges include both the endpoints and any value therebetween.

### DETAILED DESCRIPTION

The present disclosure provides formulations, methods of preparing formulations, and methods of using such formulations to be administered as dry powders for inhalation and that enable improved targeting of dry powder aerosols within the respiratory tract (e.g., to the lower respiratory tract) of patients. In some embodiments, the formulations described herein have a desired size and impaction parameter that promotes targeted delivery of formulations to peripheral regions of the lungs (e.g., small airways) and reduce the loss of drugs in the formulation to deposition in other regions of the respiratory tract (e.g., URT). For example, the extrafine carrier-based dry powder formulations described herein can bypass deposition in the URT (e.g., mouth, throat, head) and deliver dry powder formulations to the large airways and small airways, but limit deposition in the alveoli. In this way, targeted delivery of drug particles can be achieved in specific regions of the respiratory tract for improved treatment (i.e., safety and efficacy) of diseases, particularly pulmonary diseases such as asthma and chronic obstructive pulmonary disease (COPD).

There has been increasing evidence that the small airways (i.e., airways less than 2 mm in internal diameter comprising generation 8 and beyond in the respiratory tract) contribute substantially to the pathophysiologic and clinical expression of asthma and COPD. While the small airways contribute less than 10% of the overall resistance to airflow in healthy subjects, the small airways are the major site of airway obstruction in patients with asthma and COPD. The small airways also play a critical role in interstitial lung disease (e.g., idiopathic pulmonary fibrosis). The small airways are also a therapeutic target to treat inflammation in the bronchioles caused by various etiologies. Improved delivery to the small airways may also enable more effective targeting of vasodilators into the pre-capillary regions of the pulmonary arteries for the treatment of various forms of pulmonary hypertension including pulmonary arterial hypertension (PAH).

Due to the extreme variability in URT deposition observed in conventional dry powder formulations, patients may use their inhaler properly and be fully adherent with their treatment regimen, yet still receive sub-therapeutic doses of drug if the anatomical features in the soft tissue in their mouth and throat results in high URT deposition. Improving the efficiency of lung delivery (TLD) permits not just lower dose administration and reduced off-target effects, but also reduced variability associated with dose delivery to the site of action. To ensure that all patients achieve effective, targeted, dose delivery to their lungs, a feature of the improved formulations provided in this disclosure have lower dₐ and dₐ²Q values. In some embodiments, improved targeting within the lower respiratory tract enables improved delivery to the small airways, controlled release for poorly soluble drugs, and increased efficiency of systemic delivery for some less soluble or permeable APIs, features that are currently highly aspirational for inhalation products.

In some embodiments, the present disclosure provides carrier-based dry powder formulations that exhibit improved targeting to various regions within the respiratory tract upon pulmonary administration to a subject. In particular, the extrafine carrier-based dry powder formulations described herein result in decreased unwanted particle deposition in the URT, thereby increasing the total lung dose (TLD) and improving targeting of particles into the LRT and peripheral regions of the lungs (i.e., the small airways and/or alveoli). The carrier-based dry powder formulations described herein have increased precision of drug deposition within the respiratory tract and improved targeting to specific cells or receptors, thereby decreasing URT drug exposure and adverse effects, while increasing the efficiency, precision, and effectiveness of inhaled drug delivery within the LRT. Additionally, the methods described herein produce extrafine carrier-based dry powder formulations having a specific size and aerodynamic properties for efficient delivery of the drug-containing carrier particles and their respirable agglomerates to the peripheral regions of the lungs.

In some embodiments, the availability of small-particle aerosols of corticosteroids, bronchodilators, or any combinations thereof, enables a higher total lung dose deposition in specific regions of respiratory tract and better peripheral lung penetration. Improved targeting within the lower respiratory tract provides added clinical benefit (e.g., more effective treatment of inflammation in the small airways), with decreased variability in dose delivery, and diminished off-target adverse events as compared with conventional dry powder formulations.

Furthermore, traditional blends of carrier particles and drug particles require detachment of the drug particle from the carrier particles during inhalation for effective delivery into the lungs. In this respect, conventional blends must achieve a delicate balance of the adhesive forces between the drug particle and the carrier particles so that the adhesive mixture is maintained during filling and on storage, but that detachment of drug from carrier is achieved during inhalation. Otherwise, the blend will deposit in the inhaler or the URT and will not be effectively delivered to the airways. In contrast, the carrier-based dry powder formulations described herein do not require detachment of the drug particles from the carrier particles in order to be delivered with high efficiency into the lungs. Due to their small size, the carrier-based dry powder formulations described herein have very strong interparticle adhesive forces. Nonetheless, it has been surprisingly discovered that so long as the Dₐ value of the carrier particles are within the specific ranges described herein, the primary carrier particles and their agglomerates are fine enough to bypass deposition in the URT and be deposited in the lungs. Therefore, the strong adhesive forces of the particle agglomerates comprising carrier particles and drug particles do not negatively affect delivery of the carrier-based dry powder formulations. The fact that the carrier-based formulations of the present disclosure do not require the drug to be removed from the carrier to be delivered into the lungs enables nearly quantitative delivery of drug past the URT and into the LRT.

Additionally, the strong adhesive forces between drug and carrier also reduce the potential for segregation of drug from carrier during processing or in storage. Segregation of drug from carrier may lead to poor powder flow, decreased aerosol performance, and decreased uniformity in dosing. The carrier-based formulations of the present disclosure have excellent blend uniformity with no little or no particle segregation.

In some embodiments, the present disclosure also provides inhaled corticosteroids (ICS) that effectively bypass deposition in the URT, while depositing a significant fraction of the TLD in the small airways. The improved targeting of ICS reduces the potential for both local and systemic adverse events. This is especially important for pediatric asthma patients because adverse events related to the ICS often leads to poor adherence to treatment, and poor control of asthma symptoms.

The assertion that coarse particles are generally deposited in the URT while extrafine particles are deposited in the peripheral regions of the lungs neglects the significant influence of inspiratory flow rate on particle deposition. As described in the background, particle deposition in the URT and large airways is mainly driven by inertial impaction, and the impaction parameter, dₐ²Q, is a better metric for understanding regional deposition in the respiratory tract than aerodynamic diameter alone.

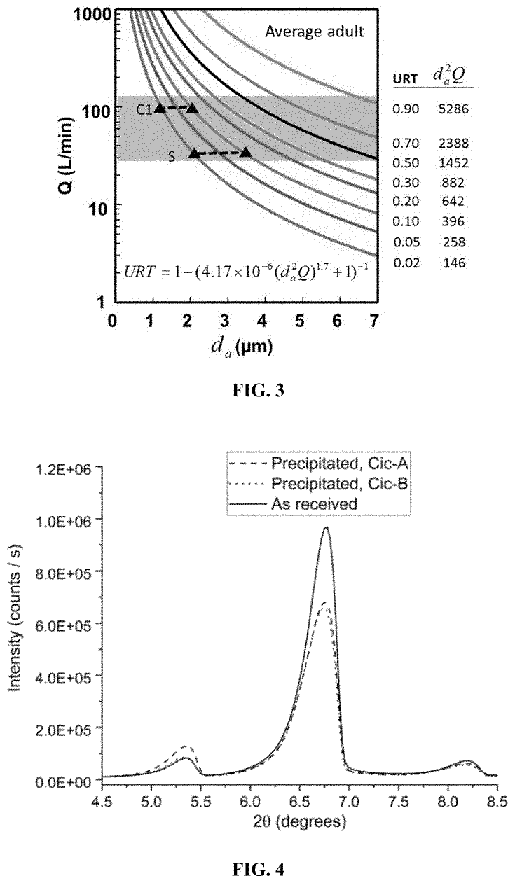

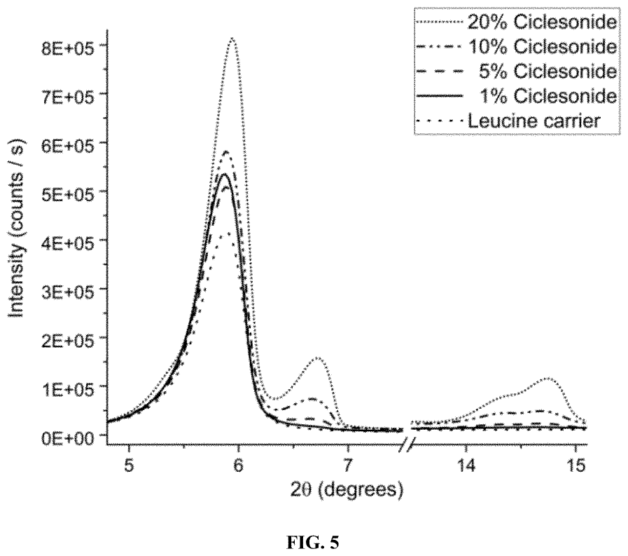

FIG. 3 replots the Stahlhofen relationship (Eq. 3) in a different fashion, detailing the combination of flow rates and aerodynamic diameters required to achieve a target impaction parameter value (dₐ²Q) and mean upper respiratory tract (URT) deposition. Accordingly, to achieve less than 10% mean URT deposition in adult subjects, the dry powder formulation, when aerosolized, should have a dₐ²Q value less than about 400 μm² L min⁻¹. In some embodiments, the dₐ²Q value is less than about 150 μm² L min⁻¹ to achieve less than 2% URT deposition. As shown in FIG. 2, achieving a dₐ²Q value<150 μm² L min⁻¹ is expected to significantly increase peripheral lung delivery. The deposition of dry powder aerosol on Stage 5 to filter (S5-F) in a NGI, provides the mass of particles with a dₐ²Q value<165 μm² L min⁻¹. As such, this stage grouping provides a good in vitro surrogate of peripheral lung delivery.

As shown in FIG. 3, there are combinations of dₐ and Q that result in a dₐ²Q value of 150 μm² L min⁻¹ or less (given by the bottom curve in FIG. 3). For example, inhalation of a 7 μm particle at a flow rate of about 3 L min⁻¹ can achieve a target dₐ²Q of 150 μm² L min⁻¹ or less. While this may be possible for a single particle, achieving this in a large ensemble of agglomerated dry powder particles is not likely, as the energy generated from such a low flow rate is insufficient to effectively disperse the particles to this aerodynamic size. At the other extreme, a dₐ²Q value of 150 μm² L min⁻¹ can be achieved for 0.4 μm particles inhaled at a flow rate of about 1000 L min⁻¹. Flow rates of this magnitude cannot be achieved by subjects with portable dry powder inhalers (DPIs). Thus, the range of practically achievable dₐ and Q values must be considered when preparing the carrier-based dry powder formulations.

The shaded area on FIG. 3 represents the range of Q values found in current marketed DPIs at a pressure drop of 4 kPa. Approximately 95% of subjects, including those with obstructive lung disease, are able to achieve pressure drops between 2 kPa and 6 kPa, with median values of about 3 kPa to 4 kPa when inhaling comfortably through a passive DPI. Within the shaded area, the range of acceptable dₐ values narrow considerably. Specific points delineated on the graph labeled Cl and S represent the flow rates for the low-resistance Concepti Inhaler, and the high-resistance Simoon™ Inhaler, respectively. FIG. 3 shows that higher resistance inhalers enable comparable URT deposition with higher values of dₐ. Modeling simulations suggest that in the absence of a 10-s breath-hold, increased particle exhalation occurs for particles with dₐ less than about 3 μm. Hence, higher resistance devices may enable low URT deposition while minimizing the potential for particle exhalation in those patients who do not perform the mandated breath-hold maneuver.

To effectively target the lungs, the inhalation device must fluidize and disperse the powder to particle sizes that enable most of the emitted dose to bypass URT deposition. This includes both primary carrier particles and agglomerates of carrier particles. For example, one path to achieving less than 2% URT deposition is based on the ensemble of carrier particles and carrier particle agglomerates having a mean dₐ value between 1.0 μm and 2.0 μm. This is markedly smaller than the range of mean dₐ values for the bimodal particle size distributions observed for marketed SPH and LB formulations (e.g., dₐ˜4.0 μm to 9.2 μm), therefore requiring novel formulation strategies.

At rest, dry powders exist as agglomerates of drug-containing particles. The dispersion energy of current DPIs is insufficient to completely disperse micronized drug from spheronized particles and large carrier particles. For both SPH and LB, these agglomerates are not a respirable size, leading to significant deposition in the device and URT. This is an inherent limitation of these types of formulations that likely cannot be overcome simply through device design. This is further evidenced by the lack of significant progress in reducing URT deposition in DPIs in the nearly 50 years since the Spinhaler® was introduced.

In some embodiments, the present disclosure provides carrier-based dry powder formulations that minimize URT deposition by utilizing extrafine particles with a low particle density, such that both the primary particles and carrier particle agglomerates remain respirable. If the target aerodynamic diameter of the bulk powder is between 1.0 μm and 2.0 μm as intimated above (for a URT deposition less than 2%), the aerodynamic size of the primary particles must be significantly less than 1.0 μm to enable particle agglomerates to achieve the target size. As discussed herein, the estimated aerodynamic diameter of the primary particles, Dₐ, was based on Eq. 4:

Dₐ=x₅₀\sqrt{ρ_{tapped}} (4)

where x₅₀ is the mass median diameter of the primary particles obtained at high dispersion pressures with a laser diffraction instrument, and ρ_{tapped} is the tapped density of the bulk powder. For carrier-free formulations comprising protein therapeutics, particles with Dₐ values between 300 and 700 nm were able to achieve TLD values >90% of the emitted dose.

A similar plot to that in FIG. 3 can be constructed for children using deposition data in the ICT model. Because of the smaller anatomical sizes in child throats (D in Eq. 1), the dₐ²Q values required to achieve comparable TLD values are much lower. In fact, achieving 10% URT deposition in the ICT requires a dₐ²Q value of about 59 μm² L min⁻¹, compared to a dₐ²Q value of 396 μm² L min⁻¹ in the AIT.

In the context of carrier-based dry powder formulations, the required Dₐ values are representative of the requirements for the carrier particles. That is, Dₐ represents the median diameter for the fully dispersed primary particles comprising the carrier powder. Ultimately, the agglomerates of the carrier particles with adhered drug or other carrier particles must remain respirable.

In some embodiments, adhesive mixtures of these extrafine carrier particles with the target Dₐ with adhered drug particles, will have a low MMIP (approximately less than 500 μ² L⁻¹ min), and as such are expected to effectively bypass deposition in the URT and be delivered with high efficiency into the LRT, and in particular, with greater efficiency into the small airways. Drug particles (micron-sized or nano-sized) that are adhered to the respirable extrafine carrier particles or agglomerates thereof, are also expected to be effectively delivered into the lungs and small airways, as the adhesive force between drug and carrier in these ‘extrafine’ formulations is expected to be strong. Unlike conventional carrier-based dry powder formulations, the carrier-based dry powder formulations provided in this disclosure do not require the drug to detach from the carrier particles for effective delivery to the lungs and small airways. Bypassing deposition in the URT is expected to also decrease interpatient variability in lung delivery that results from variations in URT deposition due to anatomical differences in the soft tissues in the mouth and throat.

In some embodiments, the ratio of particle deposition in the lower respiratory tract to that in the upper respiratory tract represents an index for lung targeting. For example, a ‘lung targeting index’ given by TLD/URT ratio can be measured in vivo by gamma scintigraphy or in vitro with the AIT or ICT throat models. In some embodiments, the TLD/URT ratio for extrafine carrier-based dry powder formulations described herein is greater than 2.0, e.g., greater than 3.0, greater than 4.0, greater than 5.0, greater than 6.0, greater than 7.0, greater than 8.0, greater than 9.0, or greater than 10.0. Conventional inhalers delivering fine drug particles have a TLD/URT ratio less than 1.0.

In some embodiments, the extrafine carrier-based dry powder formulations described herein also exhibit significantly improved regional targeting within the lungs to the lung periphery. The ‘peripheral lung index’ for airway deposition is given by the ratio of the stages in an NGI as follows: (S5-S6)/(S3-S4), with higher values representative of more peripheral deposition within the smaller airways. In some embodiments, the extrafine carrier-based dry powder formulations described herein have a peripheral lung index greater than 1.0, e.g., greater than 1.1 or greater than 1.2. In some embodiments, extrafine carrier-based dry powder formulations, expressed as a percentage of the nominal dose on stage 4 to filter (FPF_{S4-F}) of at least 40% of a nominal dose, such as greater than 50% or 60% of a nominal dose.

In some instances, it may advantageous to minimize deposition in the alveoli, i.e., by minimizing deposition on stage 7 to filter (S7-F) in an NGI. Minimizing particle deposition on S7-F may also minimize particle exhalation, as particles with an aerodynamic size of approximately 2 μm sediment about 8× more rapidly than those with an aerodynamic diameter of 0.7 μm. The ‘airway targeting index’ is given by the following ratio: (S3-S6)/(S7-F). In some embodiments, the airway targeting index may be greater than 5, e.g., greater than 10 or greater than 20, which may result in optimal airway targeting.

In some embodiments, carrier-based dry powder formulations that achieve a specific MMIP improve targeted delivery of dry powders in the respiratory tract. In some aspects, the MMIP utilizes the impaction parameter cutoffs within the NGI as opposed to the size cutoffs to define the impaction parameter distribution for particles. Flow rate independence for in vivo measurements of TLD occurs when the MMIP is constant with variations in flow rate (Weers et al., Proc Respir Drug Deliv Europe 2019, 1:59-66). Flow rate independence in vivo is not correlated with having a constant FPD_{<5 μm} with variations in flow rate.

### I. Carrier Particles

Provided herein are carrier-based dry powder formulations comprising mixtures of drug particles adhered to carrier particles. The carrier particles described herein comprise a substantially smaller geometric diameter than conventional carrier particles that can bypass deposition in the inhaler device and/or upper respiratory tract during inhalation and are respirable. In some embodiments, the carrier-based dry powder formulations described herein target drug delivery upon pulmonary administration to a subject away from the URT and into the lungs, with increased targeting into the LRT and peripheral regions of the lungs.

In contrast to the formulations provided in this disclosure, for conventional adhesive mixtures of drug and carrier (e.g., lactose blends), the gold standard for carrier particles are lactose monohydrate and other carbohydrates (e.g., mannitol). Long-chain phospholipids have also been utilized as carriers in pharmaceutical aerosols and as shell-forming excipients in carrier-free formulations. However, the complex phase behavior of these materials can lead to environmental robustness issues at high humidity. Additionally, in traditional carrier-based dry powder formulations, sometimes referred to as ‘lactose blends’, micronized drug particles are adhered to coarse lactose monohydrate carrier particles that have a geometric diameter between 60 μm and 200 μm. As such, any drug particles that remain adhered to the carrier particles will not be respirable and will deposit in the device and/or upper respiratory tract during inhalation.

In some embodiments, the carrier-based dry powder formulations described herein contain pharmaceutically acceptable crystalline carrier particles. For example, the carrier particles may have a crystallinity greater than 90%, greater than 91%, greater than 92%, greater than 93%, greater than 94%, greater than 95%, greater than 96%, greater than 97%, greater than 98%, or greater than 99%. In some embodiments, the carrier-based dry powder composition described herein utilize a low density, hydrophobic crystalline carrier with improved environmental robustness. In some embodiments, the carrier particles comprise a hydrophobic amino acid, for example, glycine, alanine, valine, leucine, isoleucine, proline, phenylalanine, methionine, and tryptophan. In some embodiments, the carrier particles are crystalline leucine carrier particles. The hydrophobic leucine particles have excellent environmental robustness with little or no difference in aerosol performance at high humidity. In some embodiments, leucine carrier particles may be selected from various isomeric or enantiomeric forms of leucine including: D-leucine, L-leucine, isoleucine, norleucine, or any combinations thereof. In some embodiments, the carrier particles are oligomers or peptides of leucine, for example, di-leucine and tri-leucine. In some instances, spray-dried leucine carrier particles with a corrugated or a porous morphology are used, and the size and density of the carrier particles can be controlled by the spray-drying process utilized to prepare them. Throughout this remainder of this disclosure, the carrier particles are often described as leucine carrier particles; however, the alternative pharmaceutically acceptable carrier particles described herein may be interchanged with leucine carrier particles in the aspects and embodiments of this disclosure.

In some embodiments, the carrier-based dry powder formulations described herein comprise hydrophobic, crystalline, leucine carrier particles with improved environmental robustness. For example, the leucine carrier particles may have improved environmental robustness relative to conventionally-used phospholipids. In some aspects, a desirable environmental robustness for the carrier is achieved when using fine (x50 ranging from 2.5 μm to 5 μm) or extrafine (x50 less than 2.5 μm) leucine carrier particles. In some embodiments, the carrier particles comprise leucine particles that are substantially crystalline (e.g., greater than 90% or greater than 95%). In some embodiments, the carrier particles are extrafine leucine particles having an x₅₀ between 0.5 μm and 2.5 μm, a tapped density between 0.01 g/cm³ and 0.30 g/cm³, and an MMIP less than 500 μm² L min⁻¹. Leucine has been extensively studied in inhaled dry powder formulations, having been utilized in carrier-based dry powder formulations as a ‘force control agent’ to modulate interparticle cohesive (drug-drug) and adhesive (drug-carrier) forces. Leucine has also been utilized as a shell-former in carrier-free formulations for inhalation. However, leucine, and the alternatives described above, have not been utilized as a carrier particle in carrier-based formulations prior to this disclosure.

In some embodiments, the carrier particle is adhered to a drug that is poorly soluble in water. In some embodiments, the carrier particle is adhered to a drug that is highly soluble in water. In both of these embodiments, the drugs are poorly soluble in a selected non-solvent. In some embodiments, the highly soluble drug is in a crystalline form. In some embodiments, the drug is in an amorphous form. In some aspects, the drug can be either highly crystalline or highly amorphous. In some aspects, the drug is not a mixture of highly crystalline and highly amorphous forms of the drug. The choice of the physical form of the drug is driven by the nature of the drug and the intended use. For example, some drugs have a higher lipophilicity, with a significantly greater molecular weight and more rotatable bonds; therefore, these drugs are difficult to crystallize and are more stable as amorphous solids.

In some embodiments, the carrier particle is a “fine” carrier particle having a median geometric diameter for the primary particles (x₅₀) between 2.5 μm and 5 μm, including, for example, between 2.5 μm and 4 μm, between 2.5 μm and 3 μm, between 3 μm and 5 μm, or between 4 μm and 5 μm.

In some embodiments, the carrier particle is a “fine” carrier particle having a tapped density between 0.03 g/cm³ and 0.40 g/cm³, e.g., 0.04 g/cm³ and 0.35 g/cm³, 0.05 g/cm³ and 0.30 g/cm³, 0.06 g/cm³ and 0.25 g/cm³, or 0.05 g/cm³ and 0.20 g/cm³.

In some embodiments, the median aerodynamic size of the primary “fine” carrier particles (Dₐ) is in the range from about 1 micron (μm) to 5 μm, e.g., from about 1.1 μm to 4.8 μm, 1.2 μm to 4.6 μm, 1.4 μm to 4.5 μm, 1.5 μm to 4.4 μm, 1.6 μm to 4.2 μm, 1.8 μm to 4 μm, 2 μm to 3.8 μm; or about 1 μm to 3 μm, 1 μm to 2.5 μm, or 1 μm to 2 μm.

In some embodiments, the adhesive mixture of “fine” carrier particles and drug particles has an MMIP between 500 and 2500 μm² L min⁻¹, e.g., from 500 μm² L min⁻ to 2250 μm² L min⁻, from 500 μm² L min⁻ to 2000 μm² L min⁻, from 550 μm² L min⁻ to 2000 μm² L min⁻, from 550 μm² L min⁻ to 1500 μm² L min⁻, from 600 μm² L min⁻ to 1250 μm² L min⁻, or from 750 μm² L min⁻ to 100 μm² L min⁻ The fine carrier particles enable improved delivery to the lungs relative to current carrier-based dry powder formulations. The fine carrier particle formulations have regional deposition that favors higher concentrations of drug in the large airways.

In some embodiments, the carrier particle is an “extrafine” carrier particle having a median geometric diameter for the primary particles (x₅₀) between 0.5 μm and 2.5 μm, including, for example, between 0.5 μm and 1.5 μm, between 0.5 μm and 1.0 μm, between 1.0 μm and 2.5 μm, or between 1.0 μm and 2.0 μm, or between 1.0 μm and 1.5 μm.

In some embodiments, the carrier particle is an “extrafine” carrier particle having a tapped density between 0.01 g/cm³ and 0.30 g/cm³, e.g., 0.02 g/cm³ and 0.20 g/cm³, 0.02 g/cm³ and 0.15 g/cm³, 0.03 g/cm³ and 0.09 g/cm³, or 0.03 g/cm³ and 0.07g/cm³.

In some aspects, the median aerodynamic size of the primary “extrafine” carrier particles (Dₐ) is less than 1000 nanometers (nm), e.g., less than 975 nm, less than 950 nm, less than 900 nm, less than 850 nm, less than 800 nm, less than 750 nm, less than 700 nm, less than 650 nm, less than 600 nm, less than 550 nm, less than 500 nm, less than 450 nm, less than 400 nm, less than 350 nm, less than 300 nm, less than 250 nm, less than 200 nm, less than 150 nm, or less than 100 nm. In some embodiments, the median aerodynamic size of the primary “extrafine” carrier particles (Dₐ) is in the range from about 300 to 700 nm, e.g., from about 350 to 700 nm, 400 to 700 nm, 450 to 700 nm, 500 to 700 nm, 550 to 700 nm, 600 to 700 nm, 650 to 700 nm; or about 300 to 650 nm, 300 to 600 nm, 300 to 550 nm, 300 to 500 nm, 300 to 450 nm, 300 to 400 nm; or about 350 to 650 nm, 350 to 600 nm, 350 to 550 nm, 350 to 500 nm, 350 to 450 nm, 350 to 400 nm; or about 400 to 650 nm, 400 to 600 nm, 400 to 550 nm, 400 to 500 nm, 400 to 550 nm; or about 500 to 650 nm, 500 to 600 nm, or 500 to 550 nm.

In some embodiments, the adhesive mixture of “extrafine” carrier particles and drug particles has an MMIP less than 500 μm²L min⁻¹, e.g., less than 450 μm²L min⁻¹, less than 400 μm² L min⁻¹, less than 350 μm² L min⁻¹, less than 300 μm² L min⁻¹, less than 250 μm² L min⁻¹, less than 200 μm² L min⁻¹, less than 150 μm² L min⁻¹, or less than 100 μm² L min⁻¹. In some embodiments, the adhesive mixture of “extrafine” carrier particles and drug particles has an MMIP from 50 μm² L min⁻ to 500 μm² L min⁻, e.g., from 60 μm² L min⁻ to 400 μm² L min⁻, from 70 μm² L min⁻ to 300 μm² L min⁻, from 80 μm² L min⁻ to 250 μm² L min⁻, from 90 μm² L min⁻ to 225 μm² L min⁻, or from 100 μm² L min⁻ to 250 μm² L min⁻. The “extrafine” carrier particles enable carrier-based dry powder formulations that effectively bypass deposition in the URT and have improved delivery to the airways, including the small airways.

In some embodiments, the carrier particles (e.g., leucine carrier particles) have a rugous surface with asperities to lower the particle density, reduce interparticle cohesive forces, and improve aerosol delivery to the lungs. In some embodiments, the leucine carrier particles have a rugosity greater than 2.0, e.g., greater than 3.0 or greater than 4.0.

In some embodiments, the required drug loading of active agent in the dry powder formulation will be the amount necessary to deliver a therapeutically effective dose of the active agent to achieve the therapeutic effect. For potent asthma/COPD therapeutics, the drug loading can be quite low, limited by there being a minimum mass of powder that needs to be filled into a receptacle to achieve the requisite accuracy and precision for delivery. In some embodiments, for the dry powder formulations of the present disclosure, the minimum fill mass is about 1 mg to 3 mg, e.g., 1 mg, 2 mg, or 3 mg. At a minimal fill mass from 1 mg to 3 mg, the drug loading is often less than 20% w/w, e.g., less than 20% w/w, 15% w/w, 12% w/w, 10% w/w, 5% w/w, 3% w/w, 1% w/w, 0.5% w/w, or 0.1% w/w.

In some embodiments, higher drug loadings may be needed for less potent drugs in other indications. There is, however, a limit to the drug loading that can be achieved in a blend before the surface of the carrier is fully saturated. In such a circumstance, excess drug may not be adhered to the carrier, but instead may be agglomerated with drug on the surface or with free drug particles. The true limit for LB may be on the order of approximately 5%. Owing to the large surface area and low density of the leucine carriers, it is likely that the acceptable drug loading may be significantly higher perhaps approaching 20% w/w or more before segregation or other forms of instability become apparent. The total lung dose of API that can be delivered with the technology of the present disclosure from a receptacle with a single inhalation is 10 mg or less. This will ultimately be dependent on the nature of the dry powder inhaler utilized and the volume of the receptacle in said inhaler. Increases in drug loading beyond approximately 5% may be expected to lead to some degree of coarsening in the aPSD.

Lactose blends (LB) and spheronized agglomerates of micronized drug (SPH) formulations were originally developed to overcome the poor powder flow properties observed with micronized drug particles. The poor powder flow led to significant variability in metering of bulk powder during filling or during metering of drug in reservoir-based dry powder inhalers. It has been surprisingly discovered that the extrafine carrier particles utilized in the present disclosure can be filled with high accuracy and precision with bespoke drum fillers, despite having poor powder flow properties.

While the utility of the nanoleucine carrier particles has been demonstrated and exemplified herein with inhaled corticosteroids (ICS), it is believed that the concepts used to design these formulations have broad utility. Virtually all drugs have limited solubility in PFOB and other fluorinated liquids used as non-solvents in the manufacturing process. As such, it is expected that nanoparticles of most drugs can be precipitated using this process. Thus, the nanoleucine carrier technology represents a platform technology for the targeted delivery of potent drugs to the airways.

### II. Formulations

Provided herein are carrier-based dry powder formulations comprising a carrier particle and an active agent. Exemplary active agents (i.e., drugs; APIs) are described in Section III of this disclosure. In some aspects, the active agent is drug particles. The drug present in the particles may be in crystalline, amorphous, or combinations thereof. For poorly soluble crystalline APIs, it may be desirable to increase the solubility and/or the dissolution rate. The formation of amorphous drug particles can lead to dramatic differences in pharmacokinetics, and as a result, differences in safety and efficacy within the lungs. In contrast, crystalline drug particles that are deposited in the lung periphery may avoid opsonization and clearance by alveolar macrophages, thereby providing a mechanism for sustaining drug within the lungs. The manufacturing process can be adjusted to control the size and physical form of APIs present in the formulation.

In some embodiments, the formulations comprise a plurality of drug particles adhered to a plurality of carrier particles. In some embodiments, one or more drug particles are adhered to a single carrier particle. In some embodiments, a single drug particle is adhered to a single carrier particle. In some embodiments, only a subset of the carrier particles are adhered to a drug particle. As described above, the number of carrier particles adhered to a drug particle depends on the drug loading and the relative sizes of the drug and carrier particles used in the formulations.

In conventional dry powder formulations, including lactose blends, the drug particles must detach from the carrier particles in order for the drug particles to be delivered into the lungs. This is because, by design, the carrier particles are too large to aerodynamically reach areas in the lungs. Thus, the drug particles must be dispersed from the carrier particles for effective delivery. In contrast, the carrier-based dry powder formulations described herein can be delivered to the lungs without detachment of the drug particles from the carrier particles. That is, the agglomerate of the carrier particles and the drug particles can reach the lungs and do not require detachment for effective delivery. It was found that the adhesive forces of the agglomerate of the carrier particles and drug particles were very strong, which would be problematic for conventional carrier-based formulations. However, due the aerodynamic size of the agglomerates of carrier particles and drug particles of the provided formulations, they can still be delivered to the large and small airways.

In some embodiments, the formulations comprise micron-sized drug particles. Thus, in some embodiments, the formulations comprise micron-sized drug particles having a x₅₀ between about 1 μm and 3 μm, including, for example, 1 μm, 1.5 μm, 2 μm, 2.5 μm, or 3 μm, or any range between the listed values. It is understood that, unless otherwise indicated, the numerical ranges provided herein include the endpoints of the range and any value in between the endpoints of the range.

In some embodiments, the formulations comprise nano-sized drug particles. In some instances, the nano-sized drug particles have at least one dimension that is less than 1000 nm. In some embodiments, the formulations comprise nano-sized drug particles having a x₅₀ less than about 1000 nanometers (nm), for example, less than 900 nm, less than 800 nm, less than 700 nm, less than 600 nm, less than 500 nm, less than 450 nm, less than 400 nm, less than 400 nm, less than 350 nm, less than 300 nm, less than 250 nm, less than 200 nm, less than 150 nm, or less than 100 nm (but greater than or equal to 1 nm). In some embodiments, the formulations comprise nano-sized drug particles having an x₅₀ between about 10 nm and 1000 nm, including, for example between 10 nm and 1000 nm, 15 nm and 750 nm, 10 nm and 500 nm, 20 nm and 450 nm, between 25 nm and 400 nm, between 50 nm and 350 nm, between 100 nm and 300 nm, between 100 nm and 250 nm, between 100 nm and 200 nm, between 100 nm and 150 nm, between 150 nm and 500 nm, between 150 nm and 450 nm, between 150 and 350 nm, between 150 and 300 nm, between 150 and 250 nm, between 150 and 200 nm, between 200 nm and 500 nm, between 200 nm and 450 nm, between 200 nm and 400 nm, between 200 nm and 350 nm, between 200 nm and 300 nm, between 200 nm and 250 nm, between 250 nm and 500 nm, between 250 nm and 450 nm, between 250 and 400 nm, between 250 and 350 nm, between 250 nm and 300 nm, between 300 nm and 500 nm, between 300 nm and 450 nm, between 300 nm and 400 nm, between 300 nm and 350 nm, between 350 nm and 500 nm, between 350 nm and 450 nm, between 350 nm and 400 nm, between 400 nm and 500 nm, or between 450 nm and 500 nm. In some embodiments, the formulations comprise nano-sized drug particles having an x₅₀ between 50 nm and 200 nm. It is understood that, unless otherwise indicated, the numerical ranges provided herein include the endpoints of the range and any value in between the endpoints of the range.

In some embodiments, the nano-sized drug particles have an x₅₀ between about 20 nm and 200 nm, e.g., about 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm or 200 nm, or any range between the listed values.

In some embodiments, all components of the drug product (i.e., drug and carrier) are present in crystalline form. In this embodiment, the carrier-based dry powder formulations described herein may be highly robust with respect to changes in humidity. This may enable use of reservoir-based multi-dose dry powder inhalers.

In some embodiments, the adhesive mixtures comprising drug particles adhered to extrafine leucine carrier particles (i.e., the “drug product”), achieve a total lung dose (TLD) is between 70 to 98% of the emitted dose (ED), for example 85 to 95% of the ED. In some embodiments, the drug product has a MMIP between 50 and 500 μm² L min⁻¹, such as between 100 μm² L min⁻¹ and 300 μm² L min⁻¹. In some embodiments, the drug product has a FPF_{S5-F} or FPF(dₐ²Q<165), representing delivery to the small airways, of 30-60% of the ED. In some embodiments, the drug product has a MMAD between 1.0 μm and 3.0 μm, such as between 1.5 μm and 2.5 μm.

The NGI is a high-performance cascade impactor used for testing portable inhalers (e.g., dry powder inhalers, metered dose inhalers, and soft mist inhalers), and nebulizers. The NGI classifies particles according to their impaction parameter. Each successive stage represents a smaller impaction parameter, which in theory enables increasingly deeper penetration within the respiratory tract. In a simple stage-grouping model, deposition in the induction port and stages 1 and 2 of the NGI is assumed to be associated with URT deposition; deposition on stages 3 and 4 with particle deposition in the large airways; deposition on stages 5 and 6 with deposition in the small airways; and deposition on stage 7 and filter (MOC) with particle deposition in the alveoli. Based on these assignments, it is possible to define two new metrics that are associated with regional deposition.

The ratio of airway to alveolar deposition, ξ, is given by the ratio of deposition on stage 3 to stage 6 to that on stage 7 to filter, i.e., (S3-S6)/(S7-F). For the purposes of this disclosure, ξ is >10, e.g., greater than 15. Increasing ξ may lead to decreases in alveolar deposition and increases in particle sedimentation rate, favoring increased deposition in the small airways and reduced particle exhalation.

The ratio of small airway to large airway deposition, ϑ, is given by the ratio of deposition of stage 5 to stage 6 to that on stage 3 to stage 4, i.e., (S5-S6/S3-S4). For the purposes of the present disclosure, ϑ is >1.0, e.g., greater than 1.5 or greater than 2.0. Higher ratios of ϑ may favor improved treatment of the small airways.

These are in vitro metrics that can be used to describe the aPSD. They are not expected to be accurate measures of the pattern of deposition for a given human subject in vivo. The deposition pattern for a given patient in vivo is influenced by many factors that cannot be reproduced in a simple in vitro model. This includes specific anatomical features of the subject, the influence of their disease on airway obstruction, the subject's inspiratory flow profile, and the numerous other mechanisms influencing particle deposition and clearance other than inertial impaction. These in vitro metrics are useful, however, in describing differences in the patterns of deposition between different formulations.

### III. Active Agent

The active agent used in the formulations and methods described herein includes an agent, drug, compound, composition of matter or mixture thereof which provides some pharmacologic, often beneficial, effect. As used herein, the terms further include any physiologically or pharmacologically active substance that produces a localized or systemic effect in a patient.

In some embodiments, any active agent that produces a localized effect in the small airways to treat diseases in the small airways can be formulated in the disclosed technology. These diseases include not only chronic obstructive lung disease and asthma, but also interstitial lung disease (e.g., idiopathic pulmonary fibrosis), and inflammation of the bronchioles (i.e., bronchiolitis) caused by various pathways including airway infections, connective tissues diseases, inflammatory bowel diseases, immune deficiencies, diffuse panbronchiolitis, and bone marrow and lung transplantation.

In some embodiments, any active agent that produces a localized effect in the systemic circulation can be formulated using the targeted formulations described herein. In some embodiments, the active agents have extensive first pass, solubility or permeability issues that limit their oral bioavailability or lead to significant variability in dosing that can be overcome with inhaled delivery.

In some embodiments, any active agent that would benefit from a rapid onset of systemic effect may benefit from the targeted formulations described herein. This would include, for example, pain medications (migraine, cluster headaches), medications for sleep disorders, or anti-anxiety medications. In some embodiments, the active agent may be for the targeted treatment of cardiac disorders (e.g., arrhythmias).

In some embodiments, an active agent for incorporation in the pharmaceutical formulation described herein may be an inorganic or an organic compound, including, without limitation, drugs which act on: the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the histamine system, and the central nervous system. Suitable active agents may be selected from, for example, hypnotics and sedatives, tranquilizers, respiratory drugs, drugs and biologics for treating asthma and COPD, anticonvulsants, muscle relaxants, anti-Parkinson agents (dopamine antagonists), analgesics, anti-inflammatories, antianxiety drugs (anxiolytics), appetite suppressants, antimigraine agents, muscle contractants, anti-infectives (antibiotics, antivirals, antifungals, vaccines) antiarthritics, antimalarials, antiemetics, anepileptics, bronchodilators, cytokines, growth factors, anti-cancer agents, antithrombotic agents, antihypertensives, cardiovascular drugs, antiarrhythmics, antioxidants, anti-asthma agents, hormonal agents including contraceptives, sympathomimetics, diuretics, lipid regulating agents, antiandrogenic agents, antiparasitics, anticoagulants, neoplastics, antineoplastics, hypoglycemics, vaccines, antibodies, diagnostic agents, and contrasting agents. The active agent, when administered by inhalation, may act locally or systemically.

The active agent may fall into one of a number of structural classes, including but not limited to small molecules, peptides, polypeptides, antibodies, antibody fragments, proteins, polysaccharides, steroids, proteins capable of eliciting physiological effects, nucleotides, oligonucleotides, polynucleotides, fats, electrolytes, and the like.

In some embodiments, the active agent may include or comprise any active pharmaceutical ingredient that is useful for treating inflammatory or obstructive airways diseases, such as asthma and/or COPD. Suitable active ingredients include long acting beta 2 agonist, such as salmeterol, formoterol, indacaterol and salts thereof, muscarinic antagonists, such as tiotropium and glycopyrronium and salts thereof, and corticosteroids including budesonide, ciclesonide, fluticasone, mometasone and salts thereof. Suitable combinations include (formoterol fumarate and budesonide), (salmeterol xinafoate and fluticasone propionate), (salmeterol xinafoate and tiotropium bromide), (indacaterol maleate and glycopyrronium bromide), and (indacaterol and mometasone). Suitable active agents also include PDE4 inhibitors, such as roflumilast and CHF6001.

In some embodiments, the active agent may include or comprise antibodies, antibody fragments, nanobodies and other antibody formats which may be used for the treatment of allergic asthma including: anti-lgE, anti-TSLP, anti-IL-5, anti-IL-4, anti-IL-13, anti-CCR3, anti-CCR-4, anti-OX40L.

In some embodiments, the active agent comprises an anti-migraine drug including rizatriptan, zolmitriptan, sumatriptan, frovatriptan or naratriptan, loxapine, amoxapine, lidocaine, verapamil, diltiazem, isometheptene, lisuride; or antihistamine drug including: brompheniramine, carbinoxamine, chlorpheniramine, azatadine, clemastine, cyproheptadine, loratadine, pyrilamine, hydroxyzine, promethazine, diphenhydramine; or anti-psychotic including olanzapine, trifluoperazine, haloperidol, loxapine, risperidone, clozapine, quetiapine, promazine, thiothixene, chlorpromazine, droperidol, prochlorperazine and fluphenazine; or sedatives and hypnotics including: zaleplon, Zolpidem, zopiclone; or muscle relaxants including: chlorzoxazone, carisoprodol, cyclobenzaprine; or stimulants including: ephedrine, fenfluramine; or antidepressants including: nefazodone, perphenazine, trazodone, trimipramine, venlafaxine, tranylcypromine, citalopram, fluoxetine, fluvoxamine, mirtazepine, paroxetine, sertraline, amoxapine, clomipramine, doxepin, imipramine, maprotiline, nortriptyline, valproic acid, protriptyline, bupropion; or analgesics including: acetaminophen, orphenadrine and tramadol; or antiemetics including: dolasetron, granisetron and metoclopramide; or opioids including: naltrexone, buprenorphine, nalbuphine, naloxone, butorphanol, hydromorphone, oxycodone, methadone, remifentanil, or sufentanil; or anti-Parkinson compounds including: benzotropine, amantadine, pergolide, deprenyl, ropinerole; or antiarrhythmic compounds including: quinidine, procainamide, and disopyramide, lidocaine, tocamide, phenyloin, moricizine, and mexiletine, flecanide, propafenone, and moricizine, propranolol, acebutolol, soltalol, esmolol, timolol, metoprolol, and atenolol, amiodarone, sotalol, bretylium, ibutilide, E-4031 (methanesulfonamide), vernakalant, and dofetilide, bepridil, nitrendipine, amlodipine, isradipine, nifedipine, nicardipine, verapamil, and diltiazem, digoxin and adenosine. Of course, active agents may comprise pharmaceutically and formulation appropriate combinations of the foregoing.

In certain embodiments, the therapeutic agent is an oncology drug, which may also be referred to as an anti-tumor drug, an anti-cancer drug, a tumor drug, an antineoplastic agent, or the like. Examples of oncology drugs that may be used include, but are not limited to, adriamycin, alkeran, allopurinol, altretamine, amifostine, anastrozole, arsenic trioxide, azathioprine, bexarotene, biCNU, bleomycin, busulfan intravenous, busulfan oral, capecitabine (Xeloda), carboplatin, carmustine, CCNU, celecoxib, chlorambucil, cisplatin, cladribine, cyclosporin A, cytarabine, cytosine arabinoside, daunorubicin, cytoxan, daunorubicin, dexamethasone, dexrazoxane, docetaxel, doxorubicin, doxorubicin, DTIC, epirubicin, estramustine, etoposide phosphate, etoposide and VP-16, exemestane, FK506, fludarabine, fluorouracil, 5-FU, gemcitabine (Gemzar), gemtuzumab-ozogamicin, goserelin acetate, hydrea, hydroxyurea, idarubicin, ifosfamide, imatinib mesylate, interferon, irinotecan (Camptostar, CPT-111), letrozole, leucovorin, leustatin, leuprolide, levamisole, litretinoin, megastrol, melphalan, L-PAM, methotrexate, methoxsalen, mithramycin, mitomycin, mitoxantrone, nitrogen mustard, paclitaxel, pamidronate, Pegademase, pentostatin, porfimer sodium, prednisone, rituxan, streptozocin, STI-571, tamoxifen, taxotere, temozolamide, teniposide, VM-26, topotecan (Hycamtin), toremifene, tretinoin, ATRA, valrubicin, velban, vinblastine, vincristine, VP16, and vinorelbine. Other examples of oncology drugs that may be used are ellipticin and ellipticin analogs or derivatives, epothilones, intracellular kinase inhibitors and camptothecins.

The active agent can be a nucleic acid, peptide, polypeptide (e.g., an antibody), cytokines, growth factors, apoptotic factors, differentiation-inducing factors, cell surface receptors and their ligands, hormones, and small molecules.

Examples of pharmaceutically active substances which may be delivered by inhalation include beta-2 agonists, steroids such as glucocorticosteroids (e.g., anti-inflammatories), anti-cholinergics, leukotriene antagonists, leukotriene synthesis inhibitors, pain relief drugs generally such as analgesics and anti-inflammatories (including both steroidal and non-steroidal anti-inflammatories), cardiovascular agents such as cardiac glycosides, respiratory drugs, anti-asthma agents, bronchodilators, anti-cancer agents, alkaloids (e.g., ergot alkaloids) or triptans such as can be used in the treatment of migraine, drugs (for instance, sulphonyl ureas) useful in the treatment of diabetes and related disorders, sleep inducing drugs including sedatives and hypnotics, psychic energizers, appetite suppressants, anti-arthritics, anti-malarials, anti-epileptics, anti-thrombotics, anti-hypertensives, anti-arrhythmics, anti-oxidants, anti-depressants, anti-psychotics, auxiolytics, anti-convulsants, anti-emetics, anti-infectives, anti-histamines, anti-fungal and anti-viral agents, drugs for the treatment of neurological disorders such as Parkinson's disease (dopamine antagonists), drugs for the treatment of alcoholism and other forms of addiction, drugs such as vasodilators for use in the treatment of erectile dysfunction or pulmonary arterial hypertension, muscle relaxants, muscle contractants, opioids, stimulants, tranquilizers, antibiotics such as macrolides, am inoglycosides, fluoroquinolones and beta-lactams, vaccines, cytokines, growth factors, hormonal agents including contraceptives, sympathomimetics, diuretics, lipid regulating agents, antiandrogenic agents, antiparasitics, anticoagulants, neoplastics, antineoplastics, hypoglycemics, nutritional agents and supplements, growth supplements, antienteritis agents, vaccines, antibodies, diagnostic agents, and contrasting agents and mixtures of the above (for example the asthma combination treatment containing both steroid and beta-agonist). More particularly, the active agent may fall into one of a number of structural classes, including but not limited to small molecules (e.g., insoluble small molecules), peptides, polypeptides, proteins, polysaccharides, steroids, nucleotides, oligonucleotides, polynucleotides, fats, electrolytes, and the like.

Specific examples include the beta-2 agonists salbutamol (e.g., salbutamol sulphate) and salmeterol (e.g., salmeterol xinafoate), the steroids budesonide and fluticasone (e.g., fluticasone propionate), the cardiac glycoside digoxin, the alkaloid anti-migraine drug dihydroergotamine mesylate and other alkaloid ergotamines, the alkaloid bromocriptine used in the treatment of Parkinson's disease, sumatriptan, rizatriptan, naratriptan, frovatriptan, almotriptan, zolmatriptan, morphine and the morphine analogue fentanyl (e.g., fentanyl citrate), glibenclamide (a sulphonyl urea), benzodiazepines such as valium, triazolam, alprazolam, midazolam and clonazepam (typically used as hypnotics, for example to treat insomnia or panic attacks), the anti-psychotic agent risperidone, apomorphine for use in the treatment of erectile dysfunction, the anti-infective amphotericin B, the antibiotics tobramycin, ciprofloxacin and moxifloxacin, nicotine, testosterone, the anti-cholinergic bronchodilator ipratropium bromide, the bronchodilator formoterol, monoclonal antibodies and the proteins LHRH, insulin, human growth hormone, calcitonin, interferon (e.g., beta- or gamma-interferon), EPO and Factor VIII, as well as in each case pharmaceutically acceptable salts, esters, analogues and derivatives (for instance prodrug forms) thereof.

Additional examples of suitable active agents include but are not limited to aspariginase, amdoxovir (RAPD), antide, becaplermin, calcitonins, cyanovirin, denileukin diftitox, erythropoietin (EPO), EPO agonists (e.g., peptides from about 10-40 amino acids in length and comprising a particular core sequence as described in WO 96/40749), dornase alpha, erythropoiesis stimulating protein (NESP), coagulation factors such as Factor VIIa, Factor VIII,

Factor IX, von Willebrand factor; ceredase, cerezyme, alpha-glucosidase, collagen, cyclosporin, alpha defensins, beta defensins, exedin-4, granulocyte colony stimulating factor (GCSE), thrombopoietin (TPO), alpha-1 proteinase inhibitor, elcatonin, granulocyte macrophage colony stimulating factor (GMCSF), fibrinogen, filgrastim, growth hormones, growth hormone releasing hormone (GHRH), GRO-beta, GRO-beta antibody, bone morphogenic proteins such as bone morphogenic protein-2, bone morphogenic protein-6, OP-1; acidic fibroblast growth factor, basic fibroblast growth factor, CD-40 ligand, heparin, human serum albumin, low molecular weight heparin (LMWH), interferons such as interferon alpha, interferon beta, interferon gamma, interferon omega, interferon tau; interleukins and interleukin receptors such as interleukin-1 receptor, interleukin-2, interluekin-2 fusion proteins, interleukin-1 receptor antagonist, interleukin-3, interleukin-4, interleukin-4 receptor, interleukin-6, interleukin-8, interleukin-12, interleukin-13 receptor, interleukin-17 receptor; lactoferrin and lactoferrin fragments, luteinizing hormone releasing hormone (LHRH), insulin, pro-insulin, insulin analogues (e.g., mono-acylated insulin as described in U.S. Pat. No. 5,922,675), amylin, C-peptide, somatostatin, somatostatin analogs including octreotide, vasopressin, follicle stimulating hormone (FSH), influenza vaccine, insulin-like growth factor (IGF), insulintropin, macrophage colony stimulating factor (M-CSF), plasminogen activators such as alteplase, urokinase, reteplase, streptokinase, pamiteplase, lanoteplase, and teneteplase; nerve growth factor (NGF), osteoprotegerin, platelet-derived growth factor, tissue growth factors, transforming growth factor-1, vascular endothelial growth factor, leukemia inhibiting factor, keratinocyte growth factor (KGF), glial growth factor (GGF), T Cell receptors, CD molecules/antigens, tumor necrosis factor (TNF), monocyte chemoattractant protein-1 endothelial growth factors, parathyroid hormone (PTH), glucagon-like peptide, somatotropin, thymosin alpha 1, thymosin alpha 1 IIb/IIIa inhibitor, thymosin beta 10, thymosin beta 9, thymosin beta 4, alpha-1 antitrypsin, phosphodiesterase (PDE) compounds, VLA-4 (very late antigen-4), VLA-4 inhibitors, bisphosponates, respiratory syncytial virus antibody, cystic fibrosis transmembrane regulator (CFTR) gene, deoxyreibonuclease (DNase), bactericidal/permeability increasing protein (BPI), and anti-CMV antibody. Exemplary monoclonal antibodies include etanercept (a dimeric fusion protein consisting of the extracellular ligand-binding portion of the human 75 kD TNF receptor linked to the Fc portion of IgGl), abciximab, afeliomomab, basiliximab, daclizumab, infliximab, ibritumomab tiuexetan, mitumomab, muromonab-CD3, iodine 131 tositumomab conjugate, olizumab, rituximab, and trastuzumab (herceptin), am ifostine, am iodarone, ambrisentan, aminoglutethimide, amsacrine, anagrelide, anastrozole, asparaginase, anthracyclines, bexarotene, bicalutamide, bleomycin, bosentan, buserelin, busulfan, cabergoline, capecitabine, carboplatin, carmustine, chlorambucin, cisplatin, cladribine, clodronate, cyclophosphamide, cyproterone, cytarabine, camptothecins, 13-cis retinoic acid, all trans retinoic acid; dacarbazine, dactinomycin, daunorubicin, dexamethasone, diclofenac, diethylstilbestrol, docetaxel, doxorubicin, epirubicin, estramustine, etoposide, exemestane, fexofenadine, fludarabine, fludrocortisone, fluorouracil, fluoxymesterone, flutamide, gemcitabine, epinephrine, L-Dopa, hydroxyurea, idarubicin, ifosfamide, imatinib, irinotecan, itraconazole, goserelin, letrozole, leucovorin, levamisole, lomustine, macitentan, mechlorethamine, medroxyprogesterone, megestrol, melphalan, mercaptopurine, methotrexate, metoclopramide, mitomycin, mitotane, mitoxantrone, naloxone, nicotine, nilutamide, octreotide, oxaliplatin, pamidronate, pentostatin, pilcamycin, porfimer, prednisone, procarbazine, prochlorperazine, ondansetron, raltitrexed, sildenafil, sirolimus, streptozocin, tacrolimus, tadalafil, tamoxifen, temozolomide, teniposide, testosterone, tetrahydrocannabinol, thalidomide, thioguanine, thiotepa, topotecan, treprostinil, tretinoin, valrubicin, vardenafil, vinblastine; vincristine, vindesine, vinorelbine, dolasetron, granisetron; formoterol, fluticasone, leuprolide, midazolam, alprazolam, amphotericin B, podophylotoxins, nucleoside antivirals, aroyl hydrazones, sumatriptan; macrolides such as erythromycin, oleandomycin, troleandomycin, roxithromycin, clarithromycin, davercin, azithromycin, flurithromycin, dirithromycin, josamycin, spiramycin, midecamycin, leucomycin, miocamycin, rokitamycin, andazithromycin, and swinolide A; fluoroquinolones such as ciprofloxacin, ofloxacin, levofloxacin, trovafloxacin, alatrofloxacin, moxifloxicin, norfloxacin, enoxacin, grepafloxacin, gatifloxacin, lomefloxacin, sparfloxacin, temafloxacin, pefloxacin, amifloxacin, fleroxacin, tosufloxacin, prulifloxacin, irloxacin, pazufloxacin, clinafloxacin, and sitafloxacin; aminoglycosides such as gentamicin, netilmicin, paramecia, tobramycin, amikacin, kanamycin, neomycin, and streptomycin, vancomycin, teicoplanin, rampolanin, mideplanin, colistin, daptomycin, gramicidin, colistimethate; polymixins such as polymixin B, capreomycin, bacitracin, penems; penicillins including penicllinase-sensitive agents like penicillin G, penicillin V; penicillinase-resistant agents like methicillin, oxacillin, cloxacillin, dicloxacillin, floxacillin, nafcillin; gram negative microorganism active agents like ampicillin, amoxicillin, and hetacillin, cillin, and galampicillin; antipseudomonal penicillins like carbenicillin, ticarcillin, azlocillin, mezlocillin, and piperacillin; cephalosporins like cefpodoxime, cefprozil, ceftbuten, ceftizoxime, ceftriaxone, cephalothin, cephapirin, cephalexin, cephradrine, cefoxitin, cefamandole, cefazolin, cephaloridine, cefaclor, cefadroxil, cephaloglycin, cefuroxime, ceforanide, cefotaxime, cefatrizine, cephacetrile, cefepime, cefixime, cefonicid, cefoperazone, cefotetan, cefmetazole, ceftazidime, loracarbef, and moxalactam, monobactams like aztreonam; and carbapenems such as imipenem, meropenem, pentamidine isethiouate, albuterol sulfate; lidocaine, metaproterenol sulfate, beclomethasone dipropionate, triamcinolone acetamide, budesonide acetonide, fluticasone, ipratropium bromide, flunisolide, cromolyn sodium, and ergotamine tartrate; taxanes such as paclitaxel; SN-38, and tyrphostins.

The methods described herein can be applied to produce micron-sized or nano-sized crystals of a poorly soluble hydrophobic drug. Examples of hydrophobic drugs include, but are not limited to, ROCK inhibitors, SYK-specific inhibitors, JAK-specific inhibitors, SYK/JAK or Multi-Kinase inhibitors, MTORs, STAT3 inhibitors, VEGFR/PDGFR inhibitors, c-Met inhibitors, ALK inhibitors, mTOR inhibitors, PI3K.delta. inhibitors, PI3K/mTOR inhibitors, p38/MAPK inhibitors, NSAIDs, steroids, antibiotics, antivirals, antifungals, antiparasitic agents, blood pressure lowering agents, cancer drugs or anti-neoplastic agents, immunomodulatory drugs (e.g., immunosuppressants), psychiatric medications, dermatologic drugs, lipid lowering agents, anti-depressants, anti-diabetics, anti-epileptics, anti-gout agents, anti-hypertensive agents, anti-malarials, anti-migraine agents, anti-muscarinic agents, anti-thyroid agents, anxiolytic, sedatives, hypnotics, neuroleptics, beta-blockers, cardiac inotropic agents, corticosteroids, diuretics, antiparkinsonian agents, gastro-intestinal agents, histamine H-receptor antagonists, lipid regulating agents, nitrates and other antianginal agents, nutritional agents, opioid analgesics, sex hormones, and stimulants.

### IV. Methods For Producing Formulations

In one aspect, the present disclosure provides methods of preparing carrier-based dry powder formulations, particularly those described in Section I of this disclosure. In some embodiments, the method for preparing the carrier-based dry powder formulations include: (a) preparation of the carrier particles with the target Dₐ values described in Section I of this disclosure; (b) preparation of drug particles; (c) homogeneous mixing of the drug particles and carrier particles in a non-solvent to form an adhesive mixture; (d) removing the liquid non-solvent to form a dry powder. In some embodiments, the active agent used for the drug particles can be one or more drugs described in Section III of this disclosure. In some embodiments, steps (b) and (c) may occur simultaneously in a single process step.

In some embodiments, a method of preparing a carrier-based dry powder formulation includes preparing extrafine leucine carrier particles with a Dₐ less than 1000 nm by spray drying a solution of leucine; adding a non-solvent to the resulting extrafine carrier particles to form a suspension; preparing a concentrated solution of drug in a solvent that is miscible with the non-solvent; adding the solution of drug to the suspension of leucine carrier particles under mixing, wherein the drug particles precipitate in the non-solvent while also forming a co-suspension with the circulating carrier particles; removing the non-solvent by lyophilization or spray drying to form a carrier-based dry powder formulation with the drug particles adhered to the extrafine leucine carrier particles (i.e., agglomerates).

In some embodiments, a method of preparing a carrier-based dry powder formulation includes preparing fine leucine carrier particles with a Dₐ between 1 μm to 5 μm by spray drying a solution of leucine; adding a non-solvent to the resulting fine carrier particles to form a suspension; preparing a concentrated solution of drug in a solvent that is miscible with the non-solvent; adding the solution of drug to the suspension of leucine carrier particles under mixing, wherein the drug particles precipitate in the nonsolvent while also forming a co-suspension with the circulating carrier particles; removing the non-solvent by lyophilization or spray drying to form a carrier-based dry powder formulation with the drug particles adhered to the fine leucine carrier particles.

### Preparation of the Carrier Particles

In some embodiments, the method of preparing a carrier-based dry powder composition includes preparing carrier particles described in Section I of this disclosure. For example, the method may include preparing extrafine carrier particles comprising a median aerodynamic diameter (Dₐ) less than 1000 nm. In some embodiments, the extrafine carrier particles comprise a Dₐ from 300 nm to 700 nm. In some embodiments, the method may include preparing fine carrier particles comprising a Dₐ from 1.0 μm to 2.5 μm. In some aspects, the Da represents the median aerodynamic diameter (Dₐ) of the primary carrier particles.

In some embodiments, fine and extrafine carrier particles may be prepared by any bottom-up manufacturing process, where the particles are precipitated to form particles of the requisite (Dₐ). In some embodiments, the bottom-up processes include spray-drying, spray freeze-drying, supercritical fluid manufacturing technologies (e.g., rapid expansion, anti-solvent, etc.), templating, microfabrication, and lithography (e.g., PRINT® technology), and other particle precipitation techniques (e.g., spinodal decomposition), for example in the presence of ultrasonic energy to ensure crystallization of the drug. In some embodiments, carrier particles are prepared using a spray-drying process. In some aspects, the spray-drying process conditions can influence the xso and surface morphology of the carrier particles.

In some embodiments, the carrier particles are comprised of leucine. In some aspects, preparing the extrafine carrier particles may include dissolving leucine in a solvent (e.g., water, ethanol, or any combinations thereof) to form a solution and spray-drying the solution under specific conditions to form extrafine leucine carrier particles comprising a Dₐ less than 1000, or fine leucine carrier particles comprising a Dₐ between 1.0 μm and 2.5 μm.

In some embodiments, the carrier particles are prepared by spray-drying a solution of leucine in water or water with a small amount of ethanol. In some embodiments, small amounts of ethanol (e.g., less than 20% w/w) can be added to an aqueous feedstock to achieve Dₐ values in the range from 100 to 500 nm. In some aspects, the spray-drying process enables control of the particle size and the particle morphology. The corrugated morphology provides low density particles with a small aerodynamic size. The spray drying process can be subdivided into smaller unit operations including: (a) feedstock preparation; (b) atomization of feedstock; (c) drying of liquid droplets; and (d) collection of dried particles. In the case of leucine particles, the nature of the atomizer and the air to liquid ratio (ALR) control the size of the atomized droplets and ultimately the size of the precipitated leucine particles. The timescale of the drying process controls the degree of crystallinity and the morphology of the particles. The addition of small amounts of ethanol (e.g., less than 20% w/w) to the aqueous feed may facilitate achievement of Dₐ values in the range from 100 to 500 nm. The spray-drying process is especially advantageous, because it enables control of not only the particle size, but also the particle morphology. The corrugated morphology provides low density particles with a small aerodynamic size. The increased rugosity of the particles decreases interparticle cohesive forces between carrier particles.

In some embodiments, the carrier precipitates as a crystalline solid during the spray-drying process. For example, hydrophobic amino acids having a molecular weight less than 200 g/mol may precipitate as crystalline solids during the spray-drying process. Owing to the low molecular weight of leucine, the amino acid precipitates as a crystalline solid during the spray-drying process. The manufacturing process involves spray-drying of a liquid feed containing dissolved leucine. For example, the spray-drying process may be performed as described in Int'l Pat. App. Pub. No. WO 2014/141069.

In some aspects, the solids content of the carrier particles in solution can influence the median aerodynamic diameter of the carrier particles. The concentration of the solids content of the carrier particles in solution may vary depending on factors including, but not limited to, the particular drugs or excipients employed in the formulation and the device to be used in the administration of the formulation. For example, batches of leucine carrier particles can be prepared from aqueous feedstocks comprising leucine dissolved in water. In this example, the solids content can affect the particle size and morphology of the leucine carrier particles. In some embodiments, the solids content (e.g., of leucine) can be from 0.4% w/w to 1.8% w/w to produce extrafine leucine carrier particles having a Dₐ from 300 nm to 700 nm. The concentration of the solids content of carrier particles may range, for example, from about 0.4% w/w and 1.8% w/w, 0.5% w/w and 1.7% w/w, from 0.6% w/w and 1.6% w/w, from 0.7% w/w and 1.5% w/w, from 0.8% w/w and 1.5% w/w, from 0.9% w/w and 1.4% w/w, or from 1.0% w/w and 1.4% w/w. In some aspects, ethanol can be added to the aqueous feedstocks comprising leucine carrier particles. It was surprisingly found that adding ethanol to the aqueous feedstock can produce extrafine leucine carrier particles having a smaller Dₐ than conventional carrier particles.

In some embodiments, the carrier particles described in Section I of this disclosure are combined with a non-solvent to form a suspension. In some embodiments, the non-solvents may comprise one or more of perfluorinated liquids (e.g., perfluorooctyl bromide, perfluorodecalin), hydrofluoroalkanes (e.g., perfluorooctyl ethane, perfluorohexyl butane, perfluorohexyl decane), hydrocarbons (e.g., octane, hexadecane), or tert-butyl alcohol. In some instances, the non-solvent is perfluorooctyl bromide (PFOB). In particular, very stable suspensions of leucine can be formed in PFOB with improved uniformity compared to some lipid suspensions. In some embodiments, the carrier particles can be substantially crystalline to improve environmental robustness. In some aspects, the carrier particles have a crystallinity greater than 90%. In some aspects, the carrier particles have a crystallinity greater than 95%.

In some embodiments, any USP Class 3 solvent (The United States Pharmacopeial Convention 2019) may be suitable as a non-solvent, provided the drug is insoluble in the liquid medium, and the leucine particles form a ‘stable’ suspension in the non-solvent. The selection of an appropriate non-solvent is dependent on the physicochemical properties of the drug substance.

### Preparation of the Drug Particles

In some embodiments, the micron-sized or nano-sized drug particles may be prepared by various top-down and bottom-up manufacturing processes. Top-down processes involve milling of coarse drug particles to form micron-sized or nano-sized drug particles. Suitable milling processes include jet milling, spiral jet milling, and media milling. Jet milling is more suitable for micron-sized particles, while media milling enables production of micron-sized or nano-sized drug particles.

As the size of the drug particles decreases, the drug particles have an increased tendency to agglomerate. In media milling, a dispersant is often used to minimize agglomerate size. Suitable dispersants include tyloxapol, long-chain phosphatidylcholines, Tween 20, or any combinations thereof.

In some embodiments, milling of crystalline drug particles can lead to the formation of amorphous domains on the surface of the milled particles. The impact of the amorphous domains on physical and chemical stability of the drug substance is molecule dependent. Minimization of amorphous content within the drug particles post-milling may be achieved in a conditioning step (e.g., recrystallization of amorphous domains at elevated humidity).

In some embodiments, the drug particles are prepared by bottom-up manufacturing processes where the drug is precipitated from solution. Suitable bottom-up processes include: spray drying, spray freeze drying, supercritical fluid processes in their various forms, templating, microfabrication, lithography (e.g., PRINT® technology), and spinodal decomposition, to name a few.

In some embodiments, the drug particles are prepared by spray drying. Detailed considerations with respect to spray drying are detailed below. The physical form of the drug following spray-drying (i.e., crystalline or amorphous) will be dependent on the molecular weight of the drug, the number of rotatable bonds of the drug and other compound structure characteristics, and the spray-drying conditions. Depending on the nature of the drug and the timescale for the drying process, the bottom-up process methods may lead to drug that is substantially crystalline (e.g., greater than 90% crystallinity) or substantially amorphous (e.g., greater than 90% amorphous) in physical form.

In some embodiments, micron-sized or nano-sized drug particles are prepared by spinodal decomposition. In this process, drug is first dissolved in a solvent that is miscible with the selected non-solvent. The drug is then precipitated by adding the drug solution dropwise into the non-solvent. In some embodiments, the rapid precipitation typically leads to amorphous nano-sized drug particles. In some aspects, the precipitated drug particles are 20 nm to 200 nm in size.

In some embodiments, the micron-sized or nano-sized drug particles created by spinodal decomposition may be nucleated and crystallized during the precipitation process by the application of ultrasonic energy. If the molecular weight of the drug is small enough, no ultrasonic energy may be required for nucleation to occur.

In some embodiments, the method may comprise preparing a solution of one or more drug(s). In some embodiments, the solution comprises a solvent that is miscible with the non-solvent. In some aspects, the solution includes a solvent comprising an alcohol (e.g., ethanol, 2-propanol), alkanes (e.g., hexane or octane), or any combination thereof. The solvent used to dissolve the drug will be dependent on the physicochemical properties of the drug. In some embodiments, when a fluorinated non-solvent is used, short-chain hydrocarbon-fluorocarbon deblocks or semi-fluorinated alkanes may be used as the solvent. These include molecules such as perfluorobulyl ethane (F₄H₂), perfluoroethyl butane (F₂H₄), and octane. In some embodiments, the solvent is a liquid at room temperature.

In some embodiments, the solvent may be a USP Class 3 solvent, such as ethanol, 1-propanol, 2-propanol, 1-butanol, 2-butanol, tert-butanol, 2-methyl-1-propanol, ethyl acetate, isopropyl acetate, isobutyl acetate, acetone, methylethylketone, methylisobutylketone, anisole, cumene, formic acid, or pentane. Depending on the physicochemical properties of the drug substance, these solvents may also be used as non-solvents in the process.

In some embodiments, the selection of the non-solvent is based on the physicochemical properties of the drug substance. The drug should not only have minimal solubility in the non-solvent, but it should also effectively disperse in the non-solvent to form a stable suspension. The solubility of the drug in the non-solvent should be less than 0.1 mg/ml, e.g., less than 0.01 mg/ml. The % Dissolved should be less than 5%, e.g., less than 1% w/w. In some embodiments, the non-solvent is a fluorinated liquid, where the fluorinated liquid is a perfluorocarbon, a halogenated fluorocarbon, or a semi-fluorinated alkane. In some embodiments the non-solvent is a perfluorinated liquid, such as perfluorooctane or perfluorodecalin. In some embodiments the non-solvent is a halogenated fluorocarbon, such as perfluorooctyl bromide, perfluorohexyl bromide, or perfluorohexyl chloride. In some embodiments the non-solvent is a semifluorinated alkane or fluorocarbon-hydrocarbon diblock, such as perfluorooctyl ethane (F8H2), perfluorohexyl ethane (F6H2), perfluorohexyl propane (F6H3), perfluorohexyl butane (F6H4), perfluorohexyl hexane (F6H6), or perfluorohexyl decane (F6H10).

In some embodiments, the preparation of the non-solvent from six carbon telomers (C6 chemistry) is beneficial due to the reduced potential to form perfluorooctanoic acid (PFOA) from the intermediate telomer iodide. The transition to the C₆ telomer chemistry requires maintaining a balance between the required physicochemical properties and the potential for increased solvency due to the shorter fluorinated chain.

### Homogeneous Mixing of the Drug and Carrier to Form an Adhesive Mixture

As the sizes of drug and carrier particles get finer, it becomes increasingly difficult to obtain uniform mixtures of the fine and extrafine carrier particles by standard high-shear and low-shear mixing processes of dry particles. Thus, in some aspects, the process is utilizes a liquid non-solvent to enable effective mixing and uniform co-suspensions of drug and fine or extrafine carrier particles.

In some embodiments, the drug particles and carrier particles are dispersed in a non-solvent. The drug particles and carrier particles form co-suspensions of agglomerates of drug and carrier. Thermodynamically, it is favorable for the drug particles to migrate away from the non-solvent, and thereby the drug particles form agglomerates with the leucine carrier particles. Alternatively, agglomerates may form when the non-solvent is removed to yield a dry powder.

In some embodiments, the drug particles and carrier particles are mixed in a non-solvent. In some embodiments, the leucine carrier particles are suspended in a non-solvent (e.g., PFOB) to form a homogeneous suspension with a high shear mixer. Under mixing conditions, the drug in solution is added dropwise to the suspension comprising the non-solvent and leucine carrier particles. The drug precipitates by spinodal decomposition as micron-sized or nano-sized drug particles to form a co-suspension. To reduce contact of the large surface area of the drug particles with the non-solvent, the drug particles form agglomerates with the circulating carrier particles. Due to the high shear mixing, the co-suspension forms a homogeneous mixture with a uniform content throughout the suspension. The relative standard deviation on dose content uniformity is less than 5%, e.g., less than 4%, less than 3%, less than 2%, or less than 1%.

In some embodiments, the carrier and drug particles can be mixed from separate non-solvent streams from a multi-headed atomizer comprising twin fluid nozzles with interacting plumes to form co-suspensions.

In some embodiments, the carrier and drug particles can be combined with a mixing nozzle to form co-suspensions.

### Removing the Liquid Non-Solvent to Form a Dry Powder

In some embodiments, after the agglomerate is formed, the non-solvent is removed. Various techniques can be employed to remove the non-solvent and to recover the dry powder formulation. In some embodiments, the non-solvent can be removed by any process that preserves the micrometric properties of the adhesive mixture of drug and carrier. Examples of techniques suitable for removing the non-solvent and recovering the dry powder formulation include, but are not limited to, evaporation, vacuum drying, spray-drying, freeze drying (lyophilization), spray freeze-drying, or any combinations thereof. In some embodiments, removing the liquid non-solvent is done by spray drying. In some embodiments, removing the liquid non-solvent is done by lyophilization.

In some embodiments, in which a non-solvent is used, it may be beneficial to recover the dry powder formulation by removing the non-solvent. For example, when the carrier and drug are particles are mixed in a non-solvent by a spinodal decomposition process or using a mixing tee or a multi-headed nozzle, the continuous liquid phase may be removed from the resulting liquid feed to obtain a dry powder. This can be done by various techniques, including spray drying and lyophilization.

### Atomization

In some embodiments, the feedstock is atomized. In one embodiment, a liquid atomizer has a structural body adapted for connection with a spray dryer and a plurality of atomizing nozzles (e.g., twin fluid nozzles). Each of the atomizing nozzles includes a liquid nozzle adapted to disperse a supply of liquid and a gas nozzle adapted to disperse a supply of gas. Exemplary atomizers with a twin fluid nozzle are described in U.S. Pat. Nos. 8,524,279 and 8,936,813. In some instances, the method comprises use of an apparatus for atomizing a liquid under dispersal conditions suitable for spray drying at a commercial plant scale.

In some embodiments, the method comprises: providing a feedstock containing an active agent in a liquid vehicle (e.g., feedstock), providing a multi-nozzle atomizer comprising a housing supporting a central gas nozzle and a plurality of atomization nozzles around the central gas nozzle, wherein each atomization nozzle comprises a liquid nozzle and a gas nozzle that is configured as a cap surrounding the liquid nozzle, and wherein the central gas nozzle is not associated with a liquid nozzle; atomizing the feedstock from the multi-nozzle atomizer to produce a droplet spray, wherein the feedstock is fed through the housing to the liquid nozzles in each of the atomization nozzles; and flowing the droplet spray in a heated gas stream to evaporate the liquid vehicle of the feedstock and produce a powder of dry particulates comprising the active agent, wherein the dry particulates have an average particle size of less than 5 microns. The active agent may comprise one or more of active agents described in Section III.

In some embodiments, significant broadening of the particle size distribution of the liquid droplets occurs above solids loading of about 1.5% w/w. The larger sized droplets in the tail of the distribution result in larger particles in the corresponding powder distribution. As a result, in some embodiments, a twin fluid nozzle is employed to generally restrict the solids loading to 1.5% w/w or less, such as 1.0% w/w, or 0.75% w/w.

In some embodiments, narrow droplet size distributions can be achieved with plane film atomizers as disclosed for example in U.S. Pat. Nos. 7,967,221 and 8,616,464 at higher solids loadings. In some embodiments, the feedstock may be atomized at solids loading between 2% and 10% w/w, such as 3% and 5% w/w. For example, an atomizer may comprise a first annular liquid flow channel, a first circular gas flow channel and a second annular gas flow channel for an atomizing gas flow, and a third gas flow channel in fluid communication with and perpendicular to said first gas flow channel. The first liquid flow channel may comprise a constriction having a diameter less than 0.51 mm (0.020 in) for spreading a liquid into a thin film in the channel. The first liquid flow channel may be intermediate to the first and second gas flow channels, and first and second gas flow channels can be positioned so that the atomizing gas impinges the liquid thin film to produce droplets. The flow of gas exiting the third gas flow channel may impinge the thin film at a right angle thereto. In some embodiments, the atomizer may be part a spray drying system. In some embodiments, the spray drying system may comprise an atomizer, a drying chamber to dry the droplets to form particles, and a collector to collect the particles.

In some embodiments, the feedstock is atomized using an atomizer with multiple twin-fluid nozzles. The plumes from the individual twin-fluid atomizers can be interacting or non-interacting.

### Drying

Drying steps may be carried out using off-the-shelf equipment used to prepare spray-dried particles for use in pharmaceuticals that are administered by inhalation. Commercially available spray-dryers include those manufactured by Büchi AG and Niro Corp.

In some embodiments, the feedstock is sprayed into a current of warm filtered air that evaporates the solvent and conveys the dried product to a collector. The spent air is then exhausted with the evaporated solvent. Operating conditions of the spray dryer such as inlet and outlet temperature, feed rate, atomization pressure, flow rate of the drying air, and nozzle configuration can be adjusted in order to produce the required particle size, moisture content, and production yield of the resulting dry particles. The selection of appropriate apparatus and processing conditions are within the purview of a skilled artisan in view of the teachings herein and may be accomplished without undue experimentation.

Exemplary settings for a NIRO® PSD-1® scale dryer (Niro Corp.) are as follows:
(i) an air inlet temperature between about 80° C. and about 200° C. (e.g., about 80, 90, 100, 110, 120, 130, 140, 150, 160, 170, 180, 190, or 200° C.) such as between about 110° C. and 170° C.;
(ii) an air outlet between about 40° C. to about 120° C. (e.g., about 40, 45, 50, 55, 60, 70, 80, 90, 100, 110, or 120° C.), such as about 60° C. and 100° C.;
(iii) a liquid feed rate between about 30 g/min to about 120 g/min (e.g., about 30, 35, 40, 45, 50, 55, 60, 65, 70, 75, 80, 85, 90, 95, 100, 110, or 120 g/min), such as about 50 g/min to 100 g/min;
(iv) total air flow of about 140 standard cubic feet per minute (scfm) to about 230 scfm (e.g., about 140, 150, 160, 170, 180, 190, 200, 210, 220, or 230 scfm), such as about 160 scfm to 210 scfm; and/or
(v) an atomization air flow rate between about 30 scfm and about 90 scfm (e.g., about 30, 40, 50, 60, 70, or 80 scfm), such as about 40 scfm to 80 scfm.

The powder population density (PPD) has been observed to correlate with primary geometric particle size. More specifically, PPD is defined as the product of solids concentration in the feedstock and liquid feed rate divided by total air flow (atomizer air plus drying air). For a given system (considering spray drying equipment and formulation), the particle size, for example, the x50 median size, of spray-dried powder is directly proportional to PPD. PPD is at least partially system dependent, therefore a given PPD number is not a universal value for all conditions. In some embodiments, a value of particle population density or PPD is between 0.01×10⁶ and 1.0×10⁶, such as between 0.03×10⁶ and 0.2×10⁶.

In some embodiments, the formulation includes anywhere from about 0.1% by weight to about 99.9% by weight active agent, e.g., from about 0.5% to about 99%, from about 1% to about 98%, from about 2% to about 95%, from about 5% to 85%, from about 10% to 80%, from about 20% to 75%, from about 25% to 70%, from about 30% to 60%, from about 35% to 55%, from about 40% to 80%, from about 40% to 70%, from about 45% to 65%, from about 50% to 90%, from about 55% to 85%, or from about 60% to 75%. In some embodiments, the amount of active agent will also depend upon the relative amounts of additives contained in the composition. In some embodiments, the compositions described herein are particularly useful for active agents that are delivered in doses of from 0.001 mg/day to 100 mg/day, or in doses from 0.01 mg/day to 75 mg/day, or in doses from 0.10 mg/day to 50 mg/day, 0.10 mg/day to 1 mg/day, 0.15 mg/day to 0.90 mg/day, or in doses from 0.20 mg/day to 40 mg/day, or in doses from 0.50 mg/day to 30 mg/day, or in doses from 1 mg/day to 25 mg/day, or in doses from 5 mg/day to 20 mg/day. It is to be understood that more than one active agent may be incorporated into the formulations described herein and that the use of the term “agent” in no way excludes the use of two or more such agents (e.g., two different drug particles or APIs). As will be understood by one of skill in the art, the incorporation of more than one active agent will depend on the nature of the device, the receptacle size, and the minimum fill mass.

### V. Delivery System

In another aspect, provided is a delivery system comprising an inhaler and the carrier-based dry powder formulations described herein. In some aspects, the carrier-based dry powder formulation is suitable for administration to the lungs via oral inhalation.

The carrier-based dry powder formulations may be formulated for use in a dry powder inhaler, such as a single use dry powder inhaler, a unit dose dry powder inhaler (e.g., capsule-based or blister-based), or a multi-dose dry powder inhaler (e.g., reservoir or blister-based).

In certain embodiments, the present disclosure is directed to a delivery system comprising a dry powder inhaler and a dry powder formulation for inhalation that comprises spray-dried particles that contains one or more active agents, wherein the in vitro total lung dose is between about 40% and 80% w/w of the nominal dose (e.g., about 40% w/w, 45% w/w, 50% w/w, 55% w/w, 60% w/w, 65% w/w, 70% w/w, 75% w/w, or 80% w/w of the nominal dose).

In some embodiments, the present disclosure is directed to a delivery system, comprising a dry powder inhaler and a dry powder formulation for inhalation that comprises spray-dried particles that contain a therapeutically active ingredient, wherein the in vitro total lung dose is between 85% and 98% w/w of the ED (e.g., about 85% w/w, 86% w/w, 87% w/w, 88% w/w, 89% w/w, 90% w/w, 91% w/w, 92% w/w, 93% w/w, 94% w/w, 95% w/w, 96% w/w, 97% w/w, or 98% w/w of the ED).

In some embodiments, suitable dry powder inhalers (DPIs) include unit dose inhalers, where the dry powder is stored in a capsule or blister, and the patient loads one or more of the capsules or blisters into the device prior to use. Alternatively, multi-dose dry powder inhalers are contemplated where the dose is pre-packaged in foil-foil blisters, for example in a cartridge, strip, or wheel. Alternatively, in some embodiments, the low hygroscopicity of powders of the present invention may enable use of reservoir-based dry powder inhalers. While any resistance of dry powder inhaler is contemplated, devices with a high device resistance (e.g., greater than 0.13 cm H₂O^{0.5} L min⁻¹) may be used to lower the flow rates, thereby reducing the inertial impaction parameter for a given sized particle.

Low resistance dry powder inhalers are generally thought to be preferred for pediatric patients to ensure that these patients generate sufficient inspiratory flow rates to effectively disperse drug from carrier. It has been demonstrated that patients inhale at higher pressure drops when using a higher resistance dry powder inhaler. High resistance inhalers typically contain dispersion elements within the device (e.g., an orifice) that improve powder dispersion, but also raise device resistance. Thus, in some embodiments, increasing device resistance may promote increased patient effort leading to more effective dose delivery in pediatric patients, despite the lower flow rate. The lower flow rate also leads to decreased impaction parameters.

Exemplary single dose dry powder inhalers include the AEROLIZER™ (Novartis, described in U.S. Pat. No. 3,991,761(Cocozza)) and BREEZHALER™ (Novartis, described in U.S. Pat. No. 8,479,730 (Ziegler et al.)). Other suitable single-dose inhalers include those described in U.S. Pat. Nos. 8,069,851 and 7,559,325.

Exemplary unit dose blister inhalers, which some patients find easier and more convenient to use to deliver medicaments requiring once daily administration, include the inhaler described in U.S. Pat. No. 8,573,197 (Axford et al).

Owing to the environmental robustness of the formulations of the present invention, it may be possible to deliver these powders with a reservoir-based DPI. Suitable DPIs include: the Turbuhaler®, Twisthaler®, Starhaler®, Genuair®, NEXThaler®, DISKUS®, Diskhaler®, to name a few.

In some embodiments, the delivery device is a breath-actuated inhaler with an oscillating actuator contained within a dispersion chamber. Examples of suitable breath actuated inhalers are described in U.S. Patent Application Publication Nos. US 2013/0340747, US 2013/0213397, and US 2016/0199598, the entire disclosures of which are incorporated by reference herein. The combination of the formulations disclosed herein and the dry powder inhalers disclosed herein enable highly efficient delivery into the lungs (TLD >70%) with high efficiency delivery into the small airways (e.g., MMIP less than 2500 μm² L⁻¹ min).

In some embodiments, the delivery device is a breath-actuated inhaler. The dry powder inhaler may include a first chamber that is adapted to receive an aerosolized powdered medicament from an inlet channel. A volume of the first chamber may be greater than a volume of the inlet channel. The dry powder inhaler may include a dispersion chamber that is adapted to receive at least a portion of the aerosolized powdered medicament from the first chamber. The dispersion chamber may hold an actuator that is movable within the dispersion chamber along a longitudinal axis. The dry powder inhaler may include an outlet channel through which air and powdered medicament exit the inhaler to be delivered to a patient. A geometry of the inhaler may be such that a flow profile is generated within the dispersion chamber that causes the actuator to oscillate along the longitudinal axis, enabling the oscillating actuator to effectively disperse powdered medicament received in the dispersion chamber for delivery to the patient through the outlet channel.

In some embodiments, the delivery device is a dry powder inhaler. The dry powder inhaler may include a powder storage region that is configured to hold a powdered medicament effective for treating exposure to particular biological and chemical agents. The inhaler may include an inlet channel. The inhaler may include a dispersion chamber that is adapted to receive air and the powdered medicament from the inlet channel. The chamber may hold an actuator that is movable within the dispersion chamber. The inhaler may include an outlet channel through which air and aerosolized medicament exit the inhaler to be delivered to a patient. A geometry of the inhaler may be such that a flow profile is generated within the dispersion chamber that causes the actuator to oscillate. This may enable the actuator when oscillating to disaggregate the powdered medicament within the dispersion chamber to be aerosolized and entrained by the air and delivered to the patient through the outlet channel.

In some instances, the dry powder inhaler may include a first chamber that is adapted to receive an aerosolized powdered medicament from an inlet channel. A volume of the first chamber may be equal to, greater than or less than the volume of the inlet channel. The dry powder inhaler may include a dispersion chamber that is adapted to receive at least a portion of the aerosolized powdered medicament from the first chamber. The dispersion chamber may hold an actuator that is movable within the dispersion chamber along a longitudinal axis. The dry powder inhaler may include an outlet channel through which air and powdered medicament exit the inhaler to be delivered to a patient. A geometry of the inhaler may be such that a flow profile is generated within the dispersion chamber that causes the actuator to oscillate along the longitudinal axis, enabling the oscillating actuator to effectively disperse powdered medicament received in the dispersion chamber for delivery to the patient through the outlet channel. During actuator oscillation, the actuator may generate an audible sound intended for feedback to the user.

### VI. Methods of Use

In one aspect, provided is a method of treating a disease in a subject comprising administering to a subject in need thereof an effective amount of a carrier-based dry powder formulation as provided in this disclosure, wherein the carrier-based dry powder formulation is administered to the subject via inhalation. The features of the formulation are described in Section I and throughout this disclosure. In some embodiments, the method comprises administering the formulation to the lungs of a subject. In some instances, the carrier-based dry powder formulation is administered as an aerosol. In some embodiments, the formulation is administered as an aerosol using an inhaler as described in Section V of this disclosure. For example, the carrier-based dry powder formulation is administered using a metered dose inhaler, a dry powder inhaler, a single dose inhaler, or a multi-unit dose inhaler. In some instances, a nebulizer or pressurized metered dose inhaler could be used.

In some embodiments, described herein is a method for the treatment of an obstructive or inflammatory airways disease, such as asthma and chronic obstructive pulmonary disease, the method comprising administering to a subject in need thereof an effective amount of the aforementioned dry powder formulation.

In some embodiments, described herein is a method for the treatment of systemic diseases, the method comprising administering to a subject in need thereof an effective amount of the aforementioned dry powder formulation.

In some embodiments, described herein is a method for delivering formulations comprising active agents as described in Section III of this disclosure (e.g., pharmaceutical drugs) to the small airways of the lungs. In order to achieve improved delivery to the small airways, the aerosolized carrier-based dry powder formulation must effectively bypass deposition in the upper respiratory tract (URT) and in the large airways, while significantly improving deposition in the small airways (e.g., generations 8 to 23). In some embodiments, the aforementioned carrier-based dry powder formulation can significantly improve deposition in generations 8 to 23 of the small airways, e.g., 8 to 20, 8 to 19, 8 to 18, 9 to 20, 10 to 18, 11 to 17, 12 to 20. In some aspects, the carrier-based dry powder formulation is deposited in generations 8 to 18 of the small airways to limit substantial deposition in the alveolar ducts and alveoli.

In some embodiments, provided is a method of aerosolizing a carrier-based dry powder formulation as provided in this disclosure. For example, the carrier-based dry powder formulation can be aerosolized using a metered dose inhaler, a dry powder inhaler, a single dose inhaler, or a multi-unit dose inhaler.

In some embodiments, the carrier-based dry powder formulation can be administered to a subject or aerosolized using an inhaler comprising a dispersion chamber having an inlet and an outlet. The dispersion chamber may include an actuator that is configured to oscillate along a longitudinal axis of the dispersion chamber. The actuator may induce air flow through the outlet channel to cause air and the carrier-based dry powder formulation to enter into the dispersion chamber from the inlet, and to cause the actuator to oscillate within the dispersion chamber to assist in dispersing the carrier-based dry powder composition from the outlet for delivery to the subject through the outlet. In some aspects, the disease is a pulmonary disease, a chronic obstructive pulmonary disease, asthma, interstitial lung disease, an airway infection, a connective tissues disease, an inflammatory bowel disease, bone marrow or lung transplantation, an immune deficiency, diffuse panbronchiolitis, bronchiolitis, or mineral dust airway disease.

In some embodiments, the carrier-based dry powder formulation comprising fine carrier particles is aerosolized for delivery to the lungs of the subject. In some embodiments, greater than 70% of the emitted dose of a carrier-based dry powder formulation comprising fine carrier particles administered to a subject is delivered to the lungs of the subject, e.g., greater than 71%, greater than 72%, greater than 73%, greater than 74%, greater than 75%, greater than 76%, greater than 77%, greater than 78%, greater than 79%, greater than 80%, greater than 81%, greater than 82%, greater than 83%, greater than 84%, greater than 85%, greater than 86%, greater than 87%, greater than 88%, greater than 89%, greater than 90%, or greater than 95%.

In some embodiments, a substantial portion (e.g., at least 70%, at least 75%, at least 80%, at least 85%, at least 90%, at least 95%, at least 96%, at least 97%, at least 98%, at least 99%, up to 100%) of the emitted dose of the carrier-based dry powder formulation comprising fine carrier particles is delivered to at least one of stages 3, 4, and 5 of a NGI (upon aerosolization of the formulation into the NGI). In some embodiments, greater than 70% of the emitted dose of the carrier-based dry powder formulation comprising fine carrier particles is delivered to at least one of stages 3, 4, and 5 of the NGI, e.g., greater than 71%, greater than 72%, greater than 73%, greater than 74%, greater than 75%, greater than 76%, greater than 77%, greater than 78%, greater than 79%, greater than 80%, greater than 81%, greater than 82%, greater than 83%, greater than 84%, greater than 85%, greater than 86%, greater than 87%, greater than 88%, greater than 89%, greater than 90%, greater than 91%, greater than 92%, greater than 93%, greater than 94%, or greater than 95%. In some aspects, a substantial portion (e.g., from 70% to 90%) of the emitted dose of the carrier-based dry powder formulation is delivered to stages 3 and 4 of the NGI, and a small residual portion (e.g., from 0% to 10%) is delivered to stage 5 of the NGI (upon aerosolization of the formulation into the NGI) .

In some embodiments, the carrier-based dry powder formulation comprising extrafine carrier particles is aerosolized for delivery to the lungs of the subject. In some aspects, greater than 90% of the emitted dose of the carrier-based dry powder formulation comprising extrafine carrier particles administered to a subject is delivered to the lungs of the subject, e.g., greater than 91%, greater than 92%, greater than 93%, greater than 94%, greater than 95 greater than 96%, greater than 97%, greater than 98%, or greater than 99%.

In some aspects, a substantial portion (e.g., at least 70%, at least 75%, at least 80%, at least 85%, at least 90%, at least 95%, at least 96%, at least 97%, at least 98%, at least 99%, up to 100%) of the emitted dose of the carrier-based dry powder formulation comprising extrafine carrier particles is delivered to at least one of stages 4, 5, and 6 of a NGI (upon aerosolization of the formulation into the NGI). In some embodiments, greater than 70% of the emitted dose of the carrier-based dry powder formulation comprising extrafine carrier particles is delivered to at least one of stages 4, 5, and 6 of the NGI, e.g., greater than 71%, greater than 72%, greater than 73%, greater than 74%, greater than 75%, greater than 76%, greater than 77%, greater than 78%, greater than 79%, greater than 80%, greater than 81%, greater than 82%, greater than 83%, greater than 84%, greater than 85%, greater than 86%, greater than 87%, greater than 88%, greater than 89%, greater than 90%, greater than 91%, greater than 92%, greater than 93%, greater than 94%, or greater than 95%.

In some aspects, a portion of the carrier-based dry powder formulation is delivered to the peripheral regions of the lungs. In some aspects, a portion of the carrier-based dry powder formulation is delivered to the lung alveoli of the subject. The carrier-based dry powder formulations as described herein enables a higher total lung deposition and better peripheral lung penetration and provides added clinical benefit, compared with large particle aerosol treatment. This may be especially beneficial in pediatric asthma patients.

In some embodiments, the small airways (airways with internal diameter <2 mm) comprise airway generations 8 to 23 and are a significant component of obstructive airway disease. Emphysema classically involves the terminal bronchioles, but it is increasingly recognized that asthma also involves small airways, not only in patients with severe asthma but also in those with milder disease. Distal airway inflammation and dysfunction also have been demonstrated in distinct clinical asthma phenotypes, such as nocturnal asthma, exercise-induced asthma, and allergic asthma. These phenotypes support the targeting of inhaled drug therapy toward the small airways.

The small airways also provide a path to the pre-capillary region of the pulmonary vasculature via the interstitial space for the treatment of other diseases, such as pulmonary arterial hypertension.

Small airway diseases that are amendable to treatment using the formulations and devices described herein include asthma, COPD, interstitial lung disease, an airway infection, a connective tissues disease, an inflammatory bowel disease, bone marrow or lung transplantation, an immune deficiency, diffuse panbronchiolitis, or a bronchiolitis selected from bronchiolitis obliterans, follicular bronchiolitis, respiratory bronchiolitis, or mineral dust airway disease.

In some cases, the delivery of carrier-based dry powder formulation comprising active agent may be more efficient than oral dose formulations by creating a high local lung concentration of the active agent, potentially yielding a quicker onset of action with likely comparable or enhanced efficacy with fewer side effects. Local delivery of active agent (e.g., APIs) directly into the lungs may circumvent poor oral bioavailability and provide even greater selectivity of effect by delivering high local lung concentrations with lower total dose exposure with the potential for greater efficacy. Administration of dry powder formulations via inhalation are also advantageous because the route of administration allows avoidance of extensive first-pass hepatic metabolism and drug-drug interaction with CYP3A inducers/inhibitors. Many drugs used to treat lung diseases can be metabolized using this enzyme system and, therefore, are susceptible to interactions or contraindications. Inhalation delivery may avoid the severity of these interactions because avoidance of first-pass metabolism, while the lower administered dose (but higher lung tissue dose) may minimize the potential for interactions. In some instances, the provided formulations have low oral and throat deposition, and a lower swallowed dose, that better targets the active agent to the ventilated areas of the lung, thereby reducing variability. By reducing the dose variability, the nominal dose of the dry powder formulation can be reduced.

In some instances, lower doses of dry powder formulations (as compared to oral dosage forms such as tablets) may be administered to a subject. In some instances, similar doses of the dry powder formulations as used for oral doses for swallowing may be administered to a subject, wherein, because the drug is administered directly to the target site, there may be a reduction in systemic drug levels when using a dry powder inhaler formulation. This may lead to a reduction of systemic toxicities associated with chronic daily use.

In some instances, pulmonary delivery with higher aerosolization efficiencies may allow less mouth and throat deposition upon aerosolization and inhalation by a subject. As mouth and throat deposited drug is swallowed and will be absorbed similarly to orally administered formulation, reducing swallowing by achieving efficient aerosolization may reduce the incidence of systemic effects.

### EMBODIMENTS

Embodiment 1: A carrier-based dry powder formulation comprising a plurality of drug particles adhered to extrafine carrier particles forming particle agglomerates having a mass median impaction parameter (MMIP) value between 50 and 500 μm² L min⁻¹.

Embodiment 2: A carrier-based dry powder formulation comprising a plurality of drug particles adhered to extrafine leucine carrier particles forming particle agglomerates having a mass median impaction parameter (MMIP) value between 50 and 500 μm²L min⁻¹.

Embodiment 3: An embodiment of any preceding or subsequent embodiment, wherein a median aerodynamic diameter of the extrafine carrier particles or the extrafine leucine carrier particles (Dₐ) is less than 1000 nm.

Embodiment 4: An embodiment of any preceding or subsequent embodiment, wherein a median aerodynamic diameter of the extrafine carrier particles or the extrafine leucine carrier particles (Dₐ) is about 300 to 700 nm.

Embodiment 5: An embodiment of any preceding or subsequent embodiment, wherein the extrafine carrier particles or the extrafine leucine carrier particles have a crystallinity greater than 90%.

Embodiment 6: A carrier-based dry powder formulation comprising a plurality of drug particles adhered to fine carrier particles forming particle agglomerates having a mass median impaction parameter (MMIP) value between 500 and 2500 μm² L min⁻¹.

Embodiment 7: A carrier-based dry powder formulation comprising a plurality of drug particles adhered to fine leucine carrier particles forming particle agglomerates having a mass median impaction parameter (MMIP) value between 500 and 2500 μm² L min⁻¹.

Embodiment 8: An embodiment of any preceding or subsequent embodiment, wherein a median aerodynamic diameter of the fine carrier particles or the fine leucine carrier particles (Dₐ) is between 1 μm and 5 μm.

Embodiment 9: An embodiment of any preceding or subsequent embodiment, wherein the fine carrier particles or the fine leucine carrier particles have a crystallinity greater than 90%.

Embodiment 10: An embodiment of any preceding or subsequent embodiment, wherein the drug particles have a mass median diameter less than 3 μm.

Embodiment 11: An embodiment of any preceding or subsequent embodiment, wherein the drug particles have a mass median diameter of about 20 nm to 500 nm.

Embodiment 12: An embodiment of any preceding or subsequent embodiment, wherein the drug particles have a crystallinity greater than 90%.

Embodiment 13: An embodiment of any preceding or subsequent embodiment, wherein the drug particles have an amorphous content greater than 90%.

Embodiment 14: An embodiment of any preceding or subsequent embodiment, wherein the drug particles comprise one or more corticosteroids, one or more bronchodilators, or any combinations thereof.

Embodiment 15: An embodiment of any preceding or subsequent embodiment, wherein the drug particles have a total lung dose in Alberta Idealized Throat of greater than 70% of an emitted dose.

Embodiment 16: An embodiment of any preceding or subsequent embodiment, wherein the drug particles have a total lung dose in Alberta Idealized Throat of greater than 90% of an emitted dose.

Embodiment 17: An embodiment of any preceding or subsequent embodiment, wherein greater than 70% of an emitted dose of the carrier-based dry powder formulation is delivered to at least one of stages 3, 4, and 5 of a NEXT GENERATION IMPACTOR™ (NGI) (upon aerosolization of the formulation into the NGI).

Embodiment 18: An embodiment of any preceding or subsequent embodiment, wherein greater than 70% of an emitted dose of the carrier-based dry powder formulation is delivered to at least one of stages 4, 5, and 6 of a NEXT GENERATION IMPACTOR™ (NGI) (upon aerosolization of the formulation into the NGI).

Embodiment 19: A method of preparing a carrier-based dry powder formulation, the method comprising: preparing carrier particles comprising a median aerodynamic diameter (Dₐ) less than 3 μm; adding a non-solvent to the carrier particles to form a suspension; preparing a drug solution comprising a drug and a solvent that is miscible with the non-solvent; adding the drug solution to the suspension of carrier particles in the non-solvent while mixing to precipitate the drug particles and thereby forming a co-suspension of drug particles and carrier particles in the non-solvent; and removing the non-solvent to form a dry powder comprising an adhesive mixture of drug particles adhered to the carrier particles, wherein the adhesive mixture has a mass median impaction parameter (MMIP) value between 50 and 2500 μm² L min⁻¹.

Embodiment 20: A method of preparing a carrier-based dry powder formulation, the method comprising: preparing an aqueous solution comprising leucine and a first solvent; drying the aqueous solution to produce fine leucine carrier particles comprising a median aerodynamic diameter (Dₐ) from 1 μm to 3 μm; adding a non-solvent to the fine leucine carrier particles to form a suspension; preparing a drug solution comprising a drug and a second solvent that is miscible with the non-solvent; adding the drug solution to the suspension of fine leucine carrier particles in the non-solvent while mixing to precipitate the drug particles and thereby forming a co-suspension of drug particles and fine leucine carrier particles in the non-solvent; and removing the non-solvent to form a dry powder comprising an adhesive mixture of drug particles adhered to the fine leucine carrier particles, wherein the adhesive mixture has a mass median impaction parameter (MMIP) value between 500 and 2500 μm² L min⁻¹.

Embodiment 21: A method of preparing a carrier-based dry powder formulation, the method comprising: preparing an aqueous solution comprising leucine and a first solvent; drying the aqueous solution to produce extrafine leucine carrier particles comprising a median aerodynamic diameter (Dₐ) less than 1000 nm; adding a non-solvent to the extrafine leucine carrier particles to form a suspension; preparing a drug solution comprising a drug and a second solvent that is miscible with the non-solvent; adding the drug solution to the suspension of extrafine leucine carrier particles in the non-solvent while mixing to precipitate the drug particles and thereby forming a co-suspension of drug particles and extrafine leucine carrier particles in the non-solvent; and removing the non-solvent to form a dry powder comprising an adhesive mixture of drug particles adhered to the extrafine leucine carrier particles wherein the adhesive mixture has a mass median impaction parameter (MMIP) value between 50 and 500 μm² L min⁻¹.

Embodiment 22: An embodiment of any preceding or subsequent embodiment, wherein the first solvent is water, ethanol, or a combination thereof.

Embodiment 23: An embodiment of any preceding or subsequent embodiment, wherein a solids content of the carrier in the first solvent is from 0.4% w/w and 1.8% w/w.

Embodiment 24: An embodiment of any preceding or subsequent embodiment, wherein a solids content of the leucine in the first solvent is from 0.4% w/w and 1.8% w/w.

Embodiment 25: An embodiment of any preceding or subsequent embodiment, wherein drying the aqueous solution to produce the carrier particles is performed by spray drying.

Embodiment 26: An embodiment of any preceding or subsequent embodiment, wherein drying the aqueous solution to produce the fine or extrafine carrier particles is performed by spray drying.

Embodiment 27: An embodiment of any preceding or subsequent embodiment, wherein drying the aqueous solution to produce the fine leucine carrier particles is performed by spray drying.

Embodiment 28: An embodiment of any preceding or subsequent embodiment, wherein drying the aqueous solution to produce the extrafine leucine carrier particles is performed by spray drying.

Embodiment 29: An embodiment of any preceding or subsequent embodiment, wherein non-solvent is a perfluorinated liquid or a fluorocarbon-hydrocarbon diblock.

Embodiment 30: An embodiment of any preceding or subsequent embodiment, wherein the non-solvent is perfluorooctyl bromide, perfluorodecalin, perfluorooctyl ethane, perfluorohexyl butane, or perfluorohexyl decane.

Embodiment 31: An embodiment of any preceding or subsequent embodiment, wherein the drug particles have a crystallinity greater than 90%.

Embodiment 32: An embodiment of any preceding or subsequent embodiment, wherein the drug solution is added dropwise to the suspension.

Embodiment 33: An embodiment of any preceding or subsequent embodiment, further comprising removing the non-solvent by spray drying the co-suspension to produce a dry powder.

Embodiment 34: An embodiment of any preceding or subsequent embodiment, further comprising removing the non-solvent by lyophilizing the co-suspension to produce a dry powder.

Embodiment 35: An embodiment of any preceding or subsequent embodiment, wherein the carrier particles have a (Dₐ) less than 3 μm and a tapped density from 0.01 g/cm³ to 0.40 g/cm³.

Embodiment 36: An embodiment of any preceding or subsequent embodiment, wherein the fine leucine carrier particles have a (Dₐ) from 1 μm than 3 μm and a tapped density from 0.05 g/cm³ to 0.40 g/cm³.

Embodiment 37: An embodiment of any preceding or subsequent embodiment, wherein the extrafine leucine carrier particles have a (Dₐ) from 300 nm to 700 nm and a tapped density from 0.01 g/cm³ to 0.30 g/cm³.

Embodiment 38: An embodiment of any preceding or subsequent embodiment, wherein the second solvent comprises 2-propanol.

Embodiment 39: An embodiment of any preceding or subsequent embodiment, wherein a blend uniformity of the drug solution in the co-suspension has a standard deviation less than 2%.

Embodiment 40: An embodiment of any preceding or subsequent embodiment, the method comprising administering to a subject in need thereof an effective amount of a carrier-based dry powder formulation of any preceding or subsequent embodiment, wherein the carrier-based dry powder formulation is administered to the subject via inhalation.

Embodiment 41: An embodiment of any preceding or subsequent embodiment, wherein the carrier-based dry powder formulation is administered as an aerosol.

Embodiment 42: An embodiment of any preceding or subsequent embodiment, wherein the carrier-based dry powder formulation is administered using a metered dose inhaler, a dry powder inhaler, a single dose inhaler, or a multi-unit dose inhaler.

Embodiment 43: An embodiment of any preceding or subsequent embodiment, wherein the carrier-based dry powder formulation is administered by providing an inhaler comprising a dispersion chamber having an inlet and an outlet, the dispersion chamber containing an actuator that is configured to oscillate along a longitudinal axis of the dispersion chamber; and inducing air flow through the outlet channel to cause air and the carrier-based dry powder formulation to enter into the dispersion chamber from the inlet, and to cause the actuator to oscillate within the dispersion chamber to assist in dispersing the carrier-based dry powder formulation from the outlet for delivery to the subject through the outlet.

Embodiment 44: An embodiment of any preceding or subsequent embodiment, wherein greater than 70% of the carrier-based dry powder formulation administered to the subject is delivered to the lungs of the subject.

Embodiment 45: An embodiment of any preceding or subsequent embodiment, wherein greater than 90% of the carrier-based dry powder formulation administered to the subject is delivered to the lungs of the subject.

Embodiment 46: An embodiment of any preceding or subsequent embodiment, wherein a portion of the carrier-based dry powder formulation is delivered to peripheral regions of the lungs of the subject.

Embodiment 47: An embodiment of any preceding or subsequent embodiment, wherein the disease is a pulmonary disease.

Embodiment 48: An embodiment of any preceding or subsequent embodiment, wherein the disease is at least one of a chronic obstructive pulmonary disease, asthma, interstitial lung disease, an airway infection, a connective tissues disease, an inflammatory bowel disease, bone marrow or lung transplantation, an immune deficiency, diffuse panbronchiolitis, bronchiolitis, or mineral dust airway disease.

### EXAMPLES

It is noted that throughout the examples leucine carrier particles are utilized; however, it is contemplated that any pharmaceutically acceptable carrier particles can be utilized.

### Example 1

### Preparation of Leucine Carrier Particles

Batches of leucine carrier particles were manufactured from aqueous feedstocks comprising leucine dissolved in water. To investigate the effect of solids content on particle size and morphology, the leucine concentration was varied between 0.3% w/w and 1.8% w/w. The feedstocks were spray dried on a Büchi B-191 spray dryer with an inlet temperature of 110° C., an outlet temperature of 65° C. to 70° C., an aspirator setting of 100%, a twin-fluid atomizer using a gas (air) pressure of 70 psi, and a liquid feed rate of 5.0 mL/min. A custom-built (Adams and Chittenden, Berkeley, CA) glass cyclone (1.75″) was used with a 1.25″ diameter×8″ long collector. Using this collection system, process yields of the leucine carrier particles are typically between 50% and 70%.

Primary particle size distributions were determined via laser diffraction (Sympatec GmbH, Clausthal-Zellerfeld, Germany). The Sympatec H3296 unit was equipped with an R2 lens, an ASPIROS micro dosing unit, and a RODOS/M dry powder-dispersing unit. Approximately 2 mg to 5 mg powder was filled into tubes, sealed and fed at 5 mm/s into a RODOS operated with 4 bar dispersion pressure and 65 mbar vacuum. Powders were introduced at an optical concentration of approximately 1% to 5% and data was collected over a measurement duration up to 15 seconds. Particle size distributions were calculated by the instrument software using the Fraünhofer model.

Tapped density was determined using a cylindrical cavity of known volume (0.593 cm³). Powder was filled into this sample holder using a microspatula. The sample cell was then gently tapped on a countertop. As the sample volume decreased, more powder was added to the cell. The tapping and addition of powder steps were repeated until the cavity was filled and the powder bed no longer consolidated with further tapping. The tapped density is defined as the mass of this tapped bed of powder divided by the volume of the cavity.

The physical properties of leucine carrier bulk powder for Examples 1-7 are presented in Table 1. Each of Examples 1-7 were prepared by the spray-drying process described above. For a leucine solids content between 0.4% w/w and 1.8% w/w, the tapped densities were comparable (0.03 g/cm³ to 0.09 g/cm³). In contrast, the particle size increased with leucine concentration, as expected. This data can be used to estimate the aerodynamic size of the primary particles that make up the bulk powder, Dₐ, as given by: Dₐ=x₅₀\sqrt{ρ_{tapped}}, where x₅₀ is the mass median diameter of the primary particles obtained at high dispersion pressures with a laser diffraction instrument and ρ_{tapped} tapped is the tapped density of the bulk powder. Equation 1 illustrates the selected approach to minimize URT deposition based on engineering extrafine particles with a low particle density, such that both the primary particles and their agglomerates remain respirable. The Dₐ values of the carrier particles of Examples 1-7 increased with leucine concentration and all were less than 1μm, ranging from 400 nm to 670 nm. Given their small size from an aerodynamic perspective, the carrier particles are hereafter referred to as “nanoleucine carrier particles.”

TABLE 1

| | Solids | Tapped | | |
|---|---|---|---|---|
| | content | density | x₅₀ | Dₐ |
| | (% w/v) | (g/cm³) | (μm) | (μm) |
| Ex. 1 | 0.4 | 0.052 | 1.76 | 0.40 |
| Ex. 2 | 0.8 | 0.051 | 2.30 | 0.52 |
| Ex. 3 | 1.3 | 0.053 | 2.87 | 0.66 |
| Ex. 4 | 1.8 | 0.043 | 3.25 | 0.67 |
| Ex. 5 | 0.3 | 0.091 | 1.86 | 0.56 |
| Ex. 6 | 1.0 | 0.038 | 2.21 | 0.43 |
| Ex. 7 | 1.7 | 0.046 | 2.70 | 0.58 |

As evidenced in Table 1, the geometric size and tapped density of the nanoleucine carrier particles differs dramatically from the characteristic values utilized in conventional adhesive mixtures comprising micronized drug particles adhered to coarse lactose carrier particles. In conventional adhesive mixtures utilizing a coarse lactose carrier particle, the x₅₀ of the coarse lactose particles is between 50 mm and 200 mm, and the tapped density is greater than 0.4 g/cm³. In conventional carrier-based dry powder formulations, the micronized drug particles are typically blended with coarse lactose carrier particles to overcome the strong interparticle cohesive forces between micronized drug particles that lead to large variability in dose delivery due to the poor powder flow properties of the fine drug particles. This is because the ratio of the cohesive forces to gravitational forces that control powder flow continues to increase as the particle size decreases. Therefore, the use of nanoleucine carrier particles described herein is outside the scope of what is generally perceived as acceptable for a carrier in formulations comprising adhesive mixtures due to the very strong adhesive forces.

### Example 2

### Feedstock Preparation of Ciclesonide Powder for Inhalation

Table 2 provides the particle properties of 1% ciclesonide/99% leucine blends prepared using nanoleucine carrier particles with different primary particle size. A feedstock for preparing an adhesive mixture of ciclesonide nanoparticles and nanoleucine carrier particles was prepared in two separate steps.

First, perfluorooctyl bromide (PFOB) was slowly added to the nanoleucine carrier particles to attain a target suspension concentration of 5% w/v. An Ultra-Turrax T10 dispersing instrument with a 5 mm dispersing tool (25000 RPM) was used to thoroughly mix the leucine particles and PFOB, resulting in a milky suspension of fine particles. Second, ciclesonide was dissolved in isopropyl alcohol (2-propanol) at a concentration of 112 mg/mL, approximately 50% of its solubility. Using an infusion pump (Harvard Apparatus, PHD 2000) coupled with a precision 1.0 mL gas-tight syringe (Hamilton 81301) with a 21-gauge needle, the ciclesonide solution was then added dropwise (infusion rate of 75 μL/min) to the stirred suspension of leucine particles to achieve a target composition of 1% ciclesonide/99% leucine. An ultrasonication probe (Sonics Vibracell, Model VC505, 3 mm stepped probe) was immersed below the location of droplet addition to provide energy for mixing as well as nucleation (operated at an amplitude of 30%).

To evaporate the combined liquid medium, the feedstock was spray-dried on a Büchi B-191 spray dryer using the collection hardware listed in Example 1. The spray-drying process parameters were: an inlet temperature of 100° C., an outlet temperature of 75 to 80° C., an aspirator setting of 100%, an atomizer gas (air) pressure of 70 psi, and a liquid feed rate of 1.0 mL/min.

The primary particle size and tapped density of the adhesive mixtures were determined using the methods described in Example 1. Assay testing was performed by weighing approximately 20 mg of formulated bulk powder onto a tared weighing paper. The weighed material was recorded and analytically transferred into a 25 mL volumetric flask following USP <1251> Method 3 to achieve an 8 μg/mL target ciclesonide concentration. The sample diluent (water: acetonitrile (50:50) (v/v)) was used to rinse the residual materials into the flask. To evaluate the uniformity of the blended nanoleucine ciclesonide powders, three independent samples were weighed as described. These samples represented different spatial locations from the container.

Quantitation of the ciclesonide content of each sample was done by reverse phase high performance liquid chromatography (RP-HPLC) with UV detection. The instrument utilized was an Agilent 1260 Infinity Series module HPLC system equipped with a UV detector. Separation was achieved with an Agilent Infinity Lab Poroshell 120 EC-C18, 3.0×150 mm, 2.7 μm column (P/N 693975-302) maintained at 40° C. and gradient separation using water:trifluoroacetic acid (0.025%, (v/v)) and Acetonitrile: Trifluoroacetic acid (0.025%, (v/v)) operated at 0.6 mL/min. The autosampler was maintained at 2-8° C. and a 40 μL injection volume was used. Ciclesonide detection was performed at 242±2 nm and quantitated by comparison to the response factor of an external standard (˜20 μg/mL drug substance). A method linearity and quantitation range of 0.08 to 200 μg/mL was established. Ciclesonide samples with a response factor greater than the reporting limit (0.05 μg/mL) were quantitated.

For all aerosol testing, size 3 hydroxypropylmethylcellulose (HPMC) clear capsules (V Caps®, Qualicaps) were hand-filled (i.e., no hand dosator was used) to achieve a 5 to 7 mg fill mass. For a 1% w/w ciclesonide powder, a target fill mass of ˜6 mg represents a 60 μg nominal dose. Aerodynamic particle size distributions (aPSD) were determined with a Next Generation Impactor (NGI) equipped with a USP induction port. No pre-separator was used since the drug-conjugated engineered nanoleucine carrier particles are respirable, with aerodynamic diameters less than 5 μm. Tests were conducted in accordance with USP <601> Aerosols ‘Aerodynamic Size Distribution, Apparatus 6 for Dry Powder Inhalers’ and Ph. Eur. 2.9.18 ‘Preparations for Inhalation; Aerodynamic Assessment of Fine Particles; Apparatus E’.

The AOS™ DPI was used for all aerosol testing. The AOS is a portable, passive, unit dose, capsule-based dry powder inhaler with a resistance of 0.051 kPa^{0.5} L⁻¹ min. aPSD tests were conducted at a pressure drop of 4 kPa, and a volume of 4 L under ambient laboratory conditions (˜20% to 40% RH). The impactor stages were coated with a solution comprising 50% v/v ethanol, 25% v/v glycerol, 22.5% v/v water and 2.5% v/v Tween 20 to prevent re-entrainment of particles within the impactor. The induction port (IP), and NGI™ stages 2 through 7 were extracted using 10 mL of sample diluent. NGI™ stages 1, 2, and MOC were extracted using 5 mL of diluent. The actuated capsule was extracted with 2 mL and the device with 5 mL of sample diluent. The ciclesonide concentration of each extract was performed per RP-HPLC, as detailed above.

Table 2 provides the particle properties of 1% ciclesonide/99% leucine blends prepared using carrier particles with different primary particle size. As shown in Table 2, the above approach was used to investigate the effect of carrier particle size. Although a larger carrier particle of Example 7 resulted in a larger x₅₀ in the blend for Example 10, the Dₐ values were insensitive to carrier particle size. The size of the particles was decreased somewhat in the manufacturing process. For Examples 8 and 10, the mean assay values were below the target composition (1% ciclesonide), which is not unusual for small batches made on lab-scale equipment. The low variability in the assay measurements, as reflected in the standard deviation (e.g., 0.01% w/w), reflects the excellent uniformity of the drug in these nanoleucine ciclesonide blends.

TABLE 2

| | | | | Assay, | Assay, | | Leucine |
|---|---|---|---|---|---|---|---|
| | | Tapped | | mean | SD | Leucine | Carrier |
| | x₅₀ | density | Dₐ | (% w/w) | (% w/w) | Carrier | x₅₀ |
| | (μm) | (g/cm³) | (μm) | (N = 3) | (N = 3) | Particle | (μm) |
| Ex. 8 | 1.66 | 0.057 | 0.40 | 0.78 | 0.01 | Ex. 5 | 1.86 |
| Ex. 9 | 1.68 | 0.052 | 0.38 | 1.05 | 0.01 | Ex. 6 | 2.21 |
| Ex. 10 | 1.98 | 0.038 | 0.39 | 0.77 | 0.01 | Ex. 7 | 2.70 |

Table 3 provides aerosol data properties of 1% ciclesonide/99% leucine blends of Examples 8-10. The batch prepared from the medium-sized carrier of Ex. 9 (utilizing carrier particles from Ex. 6) has the highest fine particle dose (FPD). In all cases, the percentage of the nominal dose retained in the capsule and device is low. For example, the capsule retention and the device retention is collectively less than 7.5% the nominal dose. Likewise, the mass of drug deposited in the USP induction port is also low.

As shown in Table 3, the fine particle dose of Examples 8-10, as measured by the drug mass on stage 4 to filter 9FPD (S4-F), of a Next Generation Impactor, is greater than 82% of the emitted dose. This data demonstrates that the 1% ciclesonide/99% leucine blends of Examples 8-10 can reach the desired target location of the small airways.

TABLE 3

| | Nominal | Capsule | Device | Throat | | | |
|---|---|---|---|---|---|---|---|
| | Dose | Retention | Retention | Deposition | FPD S4-F | FPD S4-F | MMAD |
| | (μg) | (% ND) | (% ND) | (% ND) | (μg) | (% ED) | (μm) |
| Ex. 8 | 48.27 | 1.7 | 4.9 | 3.3 | 31.64 | 82.9 | 1.96 |
| Ex. 9 | 58.30 | 1.4 | 6.1 | 2.0 | 41.81 | 89.0 | 1.95 |
| Ex. 10 | 47.43 | 0.9 | 5.1 | 2.1 | 34.47 | 82.1 | 2.19 |

### Example 3

### Neat Ciclesonide Particles

To determine whether this rapid precipitation process results in amorphous or crystalline drug, ciclesonide was dissolved in isopropyl alcohol (2-propanol) at a concentration of 112 mg/ml, approximately 50% of its solubility. Approximately 2 ml of ciclesonide solution was then added dropwise to 20 ml PFOB under constant stirring using a magnetic stir bar (1600 RPM). For one (lot Cic-B), an ultrasonication probe (Sonics Vibracell, Model VC505, 3 mm stepped probe, amplitude setting =30%) was immersed below the location of droplet addition to provide energy for mixing as well as nucleation.

Precipitation occurred spontaneously, as evident from particles accumulating at the surface of the PFOB. The precipitated ciclesonide was isolated by evaporation of the solvent (predominantly PFOB) in a vacuum oven overnight under a slow purge of dry air (25″ Hg pressure). Table 4 provides the tapped density of the precipitated ciclesonide which was determined using the methodology described in Example 1. The tapped density of the precipitated ciclesonide was between 0.16 g/cm³ and 0.17 g/cm³, about three-fold greater than that of the leucine carrier particles.

TABLE 4

| | | Tapped |
|---|---|---|
| | | density |
| | Sonication | (g/cm³) |
| Cic-A | No | 0.17 |
| Cic-B | Yes | 0.16 |

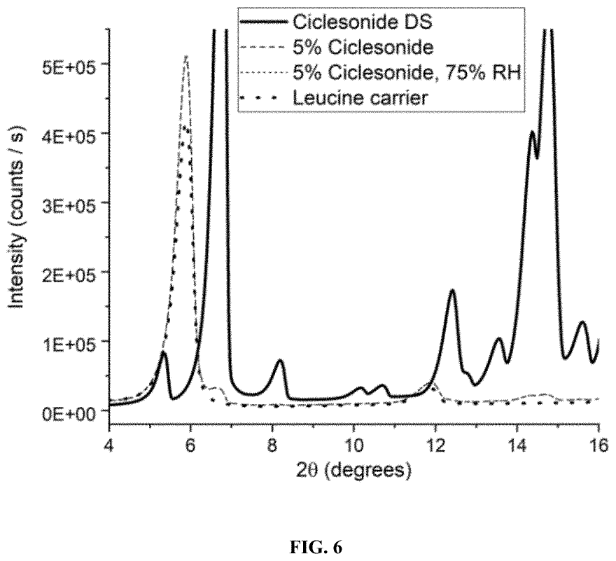

A comparison of the X-ray powder patterns of precipitated ciclesonide with that of the raw material (e.g., unprocessed starting material) is shown in FIG. 4. The positions of the peaks indicate that the precipitated material is the same physical form (polymorph) as the as-received, ciclesonide. This form has been previously reported by Feth et al. (J Pharm Sci. 2008, 97:3765-3780). This data also demonstrates the highly crystalline nature of the precipitated ciclesonide, as indicated by the lack of an amorphous background (‘halo’).

### Example 4

### Effect of Mixing Conditions on Preparation of 1% Ciclesonide Powder for Inhalation (CPI)

To assess the effect of the mixing conditions during precipitation, ciclesonide feedstocks were prepared as in Example 2, but using each of three different mixing conditions, in order from lowest to highest energy input: (1) a magnetic stir bar at 400-600 rpm, (2) an Ultra-Turrax T10 dispersing instrument with a 5 mm dispersing tool (25000 RPM), and (3) an Ultra-Turrax T10 and an ultrasonication probe (Sonics Vibracell, Model VC505, 3 mm stepped probe) operated at an amplitude of 30%. In these examples, each of the formulations used the same nanoleucine carrier particles provided in Ex. 6 (x₅₀=2.21 μm).

After addition of the ciclesonide solution to the carrier particle suspension, each feedstock was mixed with a magnetic stir bar. To evaporate the combined liquid medium, the feedstock was spray dried on a Büchi B-191 spray dryer using the collection hardware listed in Example 1. The spray-drying process parameters were as follows: an inlet temperature of 100° C., an outlet temperature of 75 to 80° C., an aspirator setting of 100%, an atomizer gas (air) pressure of 70 psi, and a liquid feed rate of 1.0 mL/min.

As provided in Table 5, the formulated CPI comprising 1% ciclesonide were characterized for primary particle size, tapped density, assay, and aPSD following the methods described in Examples 1 and 2. The primary particle size, tapped density, and Dₐ were insensitive to the mixing conditions as shown in Table 5. For each of Examples 11-13, the mean assay values were close to the target composition (e.g., 1% ciclesonide). The low variability in the assay measurements, as reflected in the standard deviation, reflects the excellent uniformity of the drug in these blends, even when prepared using low-energy mixing conditions (i.e., a magnetic stir bar).

TABLE 5

| | | | | | Assay, | Assay, |
|---|---|---|---|---|---|---|
| | | | | | mean | SD |
| | | | Tapped | | (% | (% |
| | Mixing | x₅₀ | density | Dₐ | w/w) | w/w) |
| Lot | conditions | (μm) | (g/cm³) | (μm) | (N = 3) | (N = 3) |
| Ex. 11 | Magnetic stir bar | 1.70 | 0.047 | 0.37 | 1.04 | 0.01 |
| Ex. 12 | Ultra-Turrax T10 | 1.67 | 0.051 | 0.38 | 1.07 | 0.01 |
| Ex. 13* | Ultra-Turrax T10 + | 1.68 | 0.052 | 0.38 | 1.05 | 0.01 |
| | Ultrasonication | | | | | |

*Ex. 13 utilizes the 1% ciclesonide/99% blend of Ex. 9.

Table 6 shows aerosol data properties of 1% ciclesonide/99% leucine blends prepared using the different mixing conditions described in Examples 11-13. Aerosol performance of the 1% CPI formulations prepared using different mixing conditions was assessed as described in Example 2. As shown in Table 6, the fine particle dose of Examples 11-13, as measured by the drug mass on stage 4 to filter (FPD S4-F) of a Next Generation Impactor, was greater than 89% of the emitted dose. The batch of Example 11 prepared using a magnetic stir bar for mixing had the highest FPD. In all cases, the percentage of the nominal dose retained in the capsule and device is low. Likewise, the mass of drug deposited in the throat is also low.

TABLE 6

| | Nominal | Capsule | Device | Throat | | | |
|---|---|---|---|---|---|---|---|
| | Dose | Retention | Retention | Deposition | FPD S4-F | FPD S4-F | MMAD |
| | (μg) | (% ND) | (% ND) | (% ND) | (μg) | (% ED) | (μm) |
| Ex. 11 | 50.50 | 1.2 | 1.2 | 1.7 | 40.92 | 94.5 | 1.66 |
| Ex. 12 | 57.67 | 1.1 | — | 1.6 | 43.88 | 91.5 | 1.94 |
| Ex. 13 | 58.30 | 1.4 | 6.1 | 2.0 | 41.81 | 89.0 | 1.95 |

### Example 5

### Preparation of 1, 5, 10, and 20% w/w Ciclesonide

To assess the effect of drug loading, ciclesonide feedstocks were prepared as described in Example 2, but using different amounts of drug. In all cases, the same concentration of ciclesonide in 2-propanol (approximately 112 mg/mL) was used; the drug content was controlled by varying the volume of solution infused into the stirred suspension of carrier particles. With the exception of the 5% ciclesonide composition, all formulations used leucine carrier particles prepared from a 1% w/v solution.

The carrier particle suspension was mixed with a magnetic stir bar before, during, and after addition of the ciclesonide solution. To evaporate the combined liquid medium, the feedstock was spray-dried on a Büchi B-191 spray dryer using the collection hardware listed in Example 1. The spray-drying process parameters were as follows: an inlet temperature of 100° C., an outlet temperature of 75 to 80° C., an aspirator setting of 100%, an atomizer gas (air) pressure of 70 psi, and a liquid feed rate of 1.0 mL/min.

The formulated CPI comprising 1%, 5%, 10%, and 20% ciclesonide were characterized for primary particle size, tapped density, and assay following the methods described in Examples 1 and 2. The 5%, 10% and 20% ciclesonide formulations were further diluted to achieve target ciclesonide concentrations of 10 μg/mL, 16 μg/mL, and 32 μg/mL, respectively. Table 7 shows particle properties of the ciclesonide/leucine blends with the different drug loading. Ciclesonide concentrations <10% w/w, Dₐ were found to be insensitive to drug loading. The mean and standard deviation of the assay values are discussed in Example 6.

TABLE 7

| | | | | | Assay, | Assay, |
|---|---|---|---|---|---|---|
| | | | Tapped | | mean | SD |
| | Ciclesonide | x₅₀ | density | Da | (% w/w) | (% w/w) |
| | (% w/w) | (μm) | (g/cm³) | (μm) | (N = 3) | (N = 3) |
| Example 11 | 1 | 1.70 | 0.047 | 0.37 | 1.04 | 0.01 |
| Example 14 | 5 | 2.15* | 0.045 | 0.46 | 4.75 | 0.02 |
| Example 15 | 10 | 1.74 | 0.039 | 0.34 | 11.61 | 0.11 |
| Example 16 | 20 | 2.61 | 0.103 | 0.84 | 23.66 | 0.02 |

*Carrier particles prepared from a 1.3% w/v leucine solution; all other carrier particles prepared from 1.0% w/v solution.

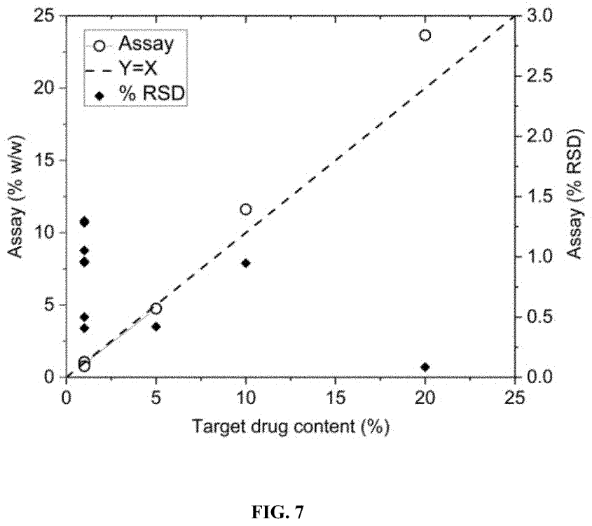

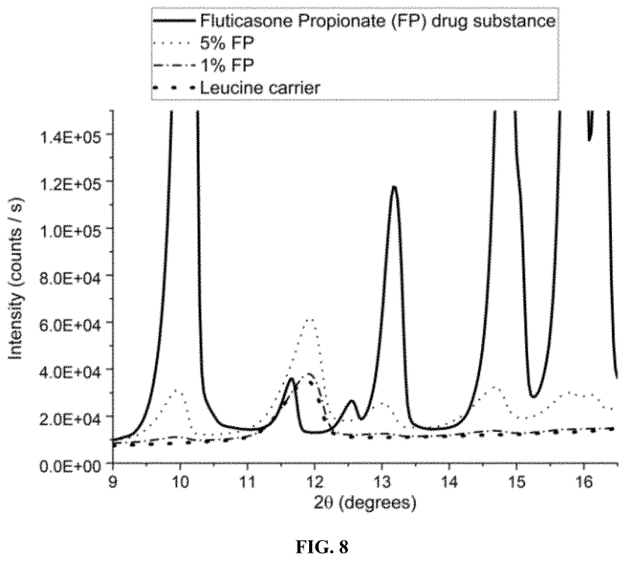

FIG. 5 shows an overlay of the X-ray powder diffraction patterns of powders comprising 1% w/w, 5% w/w, 10% w/w, and 20% w/w ciclesonide. The X-ray powder diffraction patterns for different concentrations of Examples 11 and 14-16 shows that the ciclesonide in the blends is crystalline. For example, the peak at 6.7°2θ, which could be detected for blends with a ciclesonide concentration ≥5% w/w. Upon enlargement of the powder patterns (not shown), weak diffraction peaks can be observed for the peaks at 14°2θ to 15°2θ of the 1% w/w ciclesonide powder for Example 11. For the 1% w/w blend of Example 11, the concentration of ciclesonide is near the limit of detection for the (benchtop) X-ray diffractometer used. As expected, the diffracted intensity of the ciclesonide peaks increases with drug loading. The peak positions indicate that the ciclesonide in the blend is of the same polymorph as the raw material. A qualitative assessment of the powder patterns indicates the highly crystalline nature of the blend formulation, as indicated by the lack of an amorphous background (‘halo’). However, small amounts of amorphous material are difficult to detect via changes in the broad, diffuse background. A means to detect amorphous ciclesonide is to expose the sample to elevated relative humidity (RH) and then determine if increases in the intensity of diffraction peaks are present. The 5% ciclesonide/leucine blend was exposed to 75%RH for about 20 hours, an RH sufficiently high to depress the glass transition temperature (T_{g}) of ciclesonide and induce recrystallization. As shown in FIG. 6, the XRPD patterns of Examples 11 and 14-16 before and after exposure did not change. This indicates that, within the limit of detection of the method, the ciclesonide/leucine blend contains no amorphous ciclesonide.

Table 8 shows the normalized emitted dose of CPI at different drug loadings for

Examples 11 and 14-16.

TABLE 8

| | Target | | | |
|---|---|---|---|---|
| | Ciclesonide | Emitted | Capsule | Device |
| | Content | Dose | Retention | Retention |
| | (% w/w) | (%) | (%) | (%) |
| Example 11 | 1 | 94.0 | 2.0 | 4.0 |
| Example 14 | 5 | 95.2 | 1.5 | 3.3 |
| Example 15 | 10 | 95.1 | 1.1 | 3.8 |
| Example 16 | 20 | 94.4 | 0.8 | 4.8 |

### Example 6

### Assay and Blend Uniformity

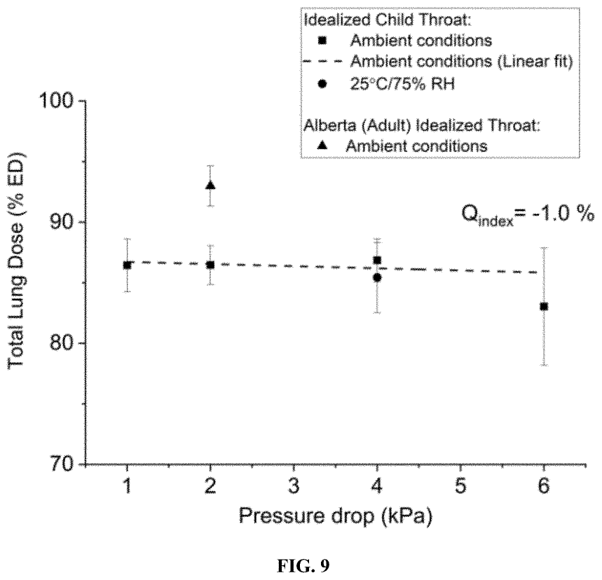

FIG. 7 shows the assay blend uniformity of the ciclesonide/leucine blends as function of the relative standard deviation (RSD). A compilation of the assay data for numerous ciclesonide blends is shown in FIG. 7. The assay results show that the drug contents of the 1% and 5% blends are close to the target content. The contents of the more concentrated blends, 10% w/w and 20% w/w, are greater than the target content. The RSD of the assay values provides a measure of the blend uniformity, as each value represents the results of three measurements on independent samples taken from different spatial areas in the powder. In all cases, the %RSD is below 1.5%, which indicates that the blends have excellent spatial homogeneity.

Achieving uniform mixing of micron-sized or nano-sized drug particles with extrafine carrier particles is difficult to achieve using low-shear or high-shear mixers. The excellent blend uniformity observed reflects the superior mixing that is achievable in a liquid-based blending process, where the carrier particles form stable suspensions in the liquid non-solvent.

Additionally, despite having significant differences in the sizes of the leucine carrier particles and the ciclesonide nanoparticles, the formulated powder exhibits little tendency to segregate in storage. This is because the interparticle adhesive forces between drug and carrier far exceed gravitational forces that would lead to segregation. Also, the cohesive forces between drug and carrier are likely to exceed dispersion forces in the inhaler, such that the drug remains adhered to the carrier during the inhalation process.

### Example 7

### Preparation of 1% w/w and 5% w/w Fluticasone Propionate Formulations

Fluticasone Propionate (FP) was dissolved in acetone at a concentration of 17 mg/ml (about 50% of the reported solubility in this solvent). Feedstocks were prepared as in Example 5. The FP content was controlled by varying the volume of solution infused into the stirred suspension of carrier particles. All formulations used leucine carrier particles prepared from a 1% w/v solution.

The carrier particle suspension was mixed with a magnetic stir bar before, during, and after addition of the FP solution. To evaporate the combined liquid medium, the feedstock was spray-dried on a Büchi B-191 spray dryer using the collection hardware listed in Example 1. The spray -drying process parameters ere: an inlet temperature of 100° C., an outlet temperature of 75 to 80° C., an aspirator setting of 100%, an atomizer gas (air) pressure of 70 psi, and a liquid feed rate of 1.0 mL/min.

The primary particle size and tapped density were determined using the methods described in Example 1. Quantitation of the fluticasone propionate content of each sample was done by reverse phase high performance liquid chromatography (RP-HPLC) with UV detection. The instrument utilized was an Agilent 1260 Infinity Series module HPLC system equipped with a UV detector. Separation was achieved with an Agilent InfinityLab Poroshell 120 EC-C18, 3.0×150 mm, 2.7 μm column (P/N 693975-302) maintained at 40° C. and gradient separation using water:trifluoroacetic acid (0.025%, (v/v)) and acetonitrile:trifluoroacetic acid (0.025%, (v/v)) operated at 0.6 mL/min. The autosampler was maintained at 2-8° C. and a 40 μL injection volume was used. Fluticasone propionate detection was performed at 238 ±2 nm and quantitated by comparison to the response factor of an external standard (˜20 μg/mL drug substance).

Table 9 shows the assay results that the drug contents of the 1% and 5% blends are close to the target content. The relative standard deviation (RSD) of the assay values provides a measure of the blend uniformity, as each value represents the results of three measurements on independent samples taken from different spatial areas in the powder. In all cases, the %RSD is below 2%, which indicates that the blends have excellent spatial homogeneity.

TABLE 9

| | | | | | Assay, | Assay, |
|---|---|---|---|---|---|---|
| | Fluticasone | | Tapped | | mean | RSD |
| | propionate | x₅₀ | density | Dₐ | (% w/w) | (% w/w) |
| | (% w/w) | (μm) | (g/cm³) | (μm) | (N = 3) | (N = 3) |
| Ex. 17 | 1 | 2.07 | 0.040 | 0.42 | 0.98 | 1.6 |
| Ex. 18 | 5 | 2.14 | 0.097 | 0.67 | 5.10 | 0.26 |

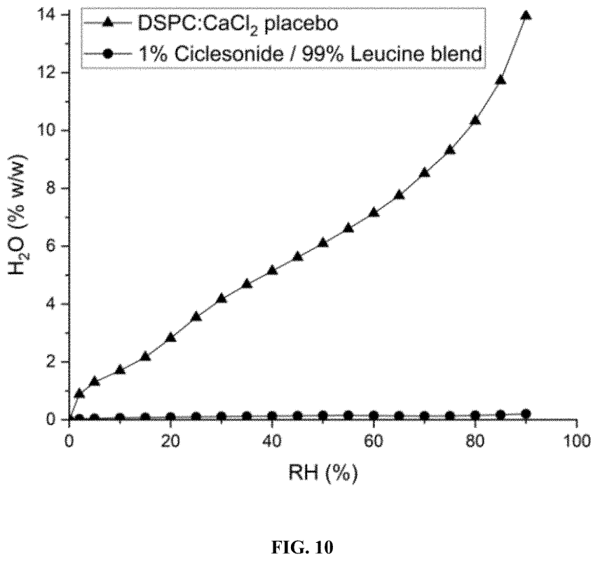

FIG. 8 shows an overlay of the X-ray powder diffraction patterns of powders comprising 1% and 5% w/w fluticasone propionate. The 5% w/w FP powder comprises crystalline fluticasone propionate which are found at the peaks at 10.0°2θ, 14.9°2θ, and 15.9°2θ. Upon enlargement of the powder pattern (not shown), weak peaks can be observed at above the peak positions for the 1% w/w FP powder. As was observed for ciclesonide, the diffracted intensity of the fluticasone peaks in the 1% w/w FP blend is near the limit of detection for the (benchtop) X-ray diffractometer used.

### Example 8

### Ciclesonide Powder for Inhalation

In the examples that follow, comparisons will be made for Example 11 of ciclesonide powder for inhalation with various marketed inhaled corticosteroid (ICS) formulations. The physicochemical properties of 1% ciclesonide powder for inhalation of Example 11 are detailed in

Table 10. The aerosol properties of Example 11 are detailed in Table 11.

TABLE 10

| Metric | Mean |
|---|---|
| Ciclesonide content (% w/w) | 0.96 |
| Blend uniformity (% RSD) | 1.04 |
| Geometric size × 10 (μm) | 0.79 |
| × 50 (μm) | 1.70 |
| × 90 (μm) | 3.16 |
| Tapped density (g/cm³) | 0.047 |
| Primary aerodynamic diameter, Dₐ (μm) | 0.37 |
| Water content, DVS (% w/w) | <0.3 |
| ICS physical form, XRPD | Crystalline |
| Leucine physical form, XRPD | Crystalline |

TABLE 11

| Metric | Mean |
|---|---|
| Emitted dose, ED (% nominal dose) ^{a} | 94.0 |
| Fine particle dose < 5 μm, | 96.8 |
| FPD_{<5μm}, (% emitted dose) ^{b} | |
| Fine particle dose S4-F, FPD_{S4-F} (% emitted dose) ^{b} | 94.5 |
| Mass median aerodynamic diameter, MMAD (μm) ^{b} | 1.66 |
| Geometric standard deviation, GSD ^{b} | 1.57 |
| Mass median impaction parameter, | 115.8 |
| MMIP (μm² L/min) ^{b} | |
| Total lung dose, TLD [AIT] (% emitted dose) ^{a} | 93.0 |
| Total lung dose, TLD [ICT] (% emitted dose) ^{a} | 86.5 |
| Q index (%) ^{a} | −1.0 |
| Humidity dependence, TLD_{75% RH}/TLD_{40% RH} ^{b} | 0.99 |

^{a} ΔP = 2 kPa, Vi = 2 L
^{b} ΔP = 4 kPa, Vi = 4 L

### Example 9

### Flow Rate Independence and Environmental Robustness of CPI

The total lung dose of a 1% ciclesonide formulation (Example 11) prepared in Example 4 was assessed using two anatomical throat models, the Alberta Idealized Throat (AIT) and the Idealized Child Throat (ICT). These models were developed by Finlay et al. at the University of Alberta using CT or MRI scans to provide particle deposition patterns mimicking an average adult and child, respectively.

The AOS DPI was coupled to the inlet of the AIT/ICT model using a custom mouthpiece adaptor (MSP Corporation, USA). The dose bypassing the throat was collected downstream on a 76 mm diameter filter A/E type glass fiber 1 μm, (Pall Corp., US) mounted in the filter housing of the Fast Screening Impactor, FSI (MSP Corporation, USA). The interior surfaces of the throat were coated with 15 mL of a solution comprising 50% v/v methanol and 50% v/v Tween 20 to mimic the hydrated oropharyngeal mucosa and to prevent particle resuspension. The coating solution was allowed to wet the internal walls of the AIT using a rocking or rotary motion to tilt the throat from side to side. Excess coating solution was allowed to drain for 5 min before use.

For determination of in-vitro TLD, a filled capsule (˜6 mg fill mass; target 60 μg ciclesonide) was loaded into the AOS DPI inhaler and punctured. A Copley model TPK2001 critical flow controller, and Copley model HCPS vacuum pump was activated. This draws air at the desired pressure drop through the inhaler for a total volume of 2 L, depositing the TLD on the filter. The filter was removed from the Fast Screening Impactor, placed in a plastic bag, then extracted using 20 mL sample diluent (water:acetonitrile (50:50 (v/v)).

The total lung dose of a 1% ciclesonide formulation (Example 11) prepared in Example 4 was assessed. Capsules were hand-filled (i.e., no hand dosator was used) to achieve a 5 to 7 mg fill mass. For this ciclesonide powder, a target fill mass of ˜6 mg represents a 60 μg nominal dose. The AOS™ DPI was used for all aerosol testing, as described in Example 2.

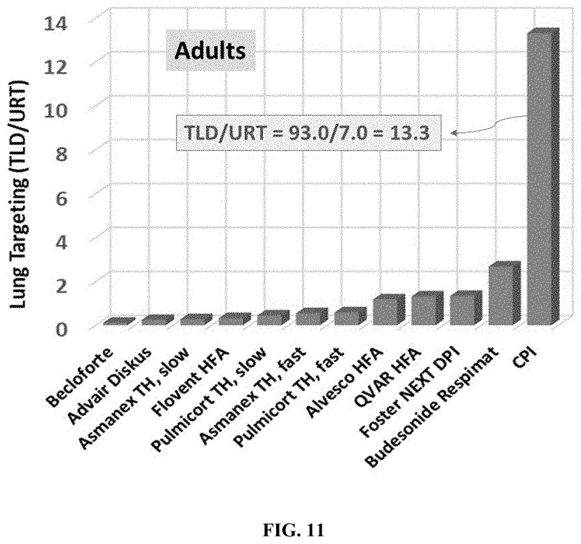

The total lung dose (TLD) is given by the mass of drug that bypasses either an Idealized Child Throat (ICT) or an Alberta Idealized (adult) Throat (AIT). As reported here, this dose is normalized by mass of drug emitted from the device (FIG. 9).

TLD performance of CPI batch of Example 11 in the ICT and AIT models are presented in Table 11. The TLD was 93.0% in the AIT and 86.5% in the ICT.

FIG. 9 shows the TLD performance of CPI batch of Example 11 in the ICT model was evaluated at a 1 kPa, 2 kPa, 4 kPa, and 6 kPa pressure drops and 2 L volume. There was little change in TLD over this range of pressure drops. One metric for quantitating the degree of flow rate dependence is termed the Q index, which is derived from a linear regression of a plot of TLD vs. ΔP. It represents the percent difference in TLD between pressure drops of 6 and 1 kPa normalized by the higher of the two TLD values. This range of pressure drops encompasses what most patients achieve when utilizing DPIs. We define low flow rate dependence as having a |Q index| between 0 and 15%, medium flow rate dependence as having a |Q index| between 15 and 40%, and high flow rate dependence as having a |Q index|>40%.

Dispersion of the drug from the carrier or spheronized agglomerate depends critically on the pressure drop that patients achieve through their dry powder inhaler during inhalation. This is often referred to as flow rate dependence. The ability to achieve acceptable inspiratory pressures is dependent on the age of the patient. Pediatric and geriatric patients have reduced muscle strength, and sometimes may be unable to generate the inspiratory pressures needed to achieve effective drug dispersion.

Given that the Q index of the TLD vs. AP data in the ICT is only −1.0% (FIG. 6), the 1% ciclesonide blend aerosolized using the AOS DPI has low flow rate dependence, or even flow rate independence.

TLD determinations were also performed at elevated RH (75%) to assess the effect of humidity on aerosol performance. Environmental robustness of 1% CPI batch of Example 11 was performed by placing the identical ICT test apparatus, as described, into an environmental chamber (Barnsted International, Model EC12560) operated at 75% RH. The TLD using the AOS DPI and ICT was performed at 4 kPa pressure drop and 2 L volume using the same configuration and methodology as described above. The ciclesonide concentration of each extract was performed per RP-HPLC, as detailed in Example 2 above and reported in terms of % of the total recovered dose relative to the average emitted dose. The data measured in the ICT at elevated RH (25° C./75% RH) illustrates that this drug-device combination has excellent environmental robustness. This is not surprising given the highly crystalline, hydrophobic nature of the drug and carrier.

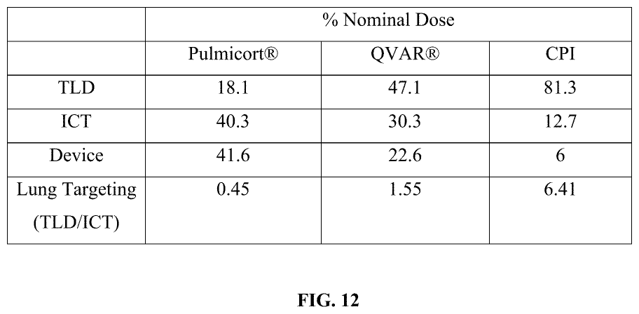

A highly crystalline formulation is expected to provide an advantage with respect to the environmental robustness of aerosol performance. Highly crystalline materials tend to be non-hygroscopic, taking up very little water even at elevated relative humidity conditions is a comparison of the moisture sorption isotherms of a 1% ciclesonide/leucine blend (Example 11) and a spray-dried ‘benchmark’ carrier, DSPC:CaCl₂ (FIG. 10). This carrier particle comprises about 93% w/w distearoylphosphatidylcholine, a phospholipid considered to be hydrophobic. Overall, the moisture uptake of the ciclesonide/leucine blend is low; at the highest RH, the water content is only 0.2% w/w. In contrast, the DSPC:CaCl₂ placebo is considerably more hygroscopic. At any RH, the DSPC:CaCl₂ placebo is between 30 and 80 times more hygroscopic than the ciclesonide/leucine blend.

### Example 10

### Targeting of Inhaled Corticosteroids to the Lungs: Comparison to Current Marketed ICS

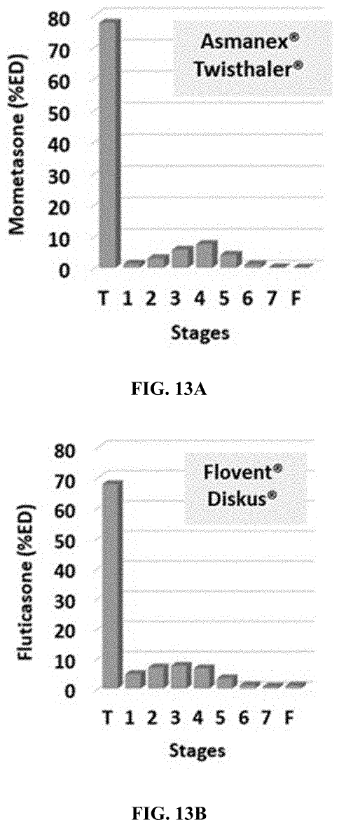

Example 11 (ciclesonide powder for inhalation, CPI), as detailed in Example 8 (Tables 10 and 11) improves targeting of ICS to the lungs of adults relative to current marketed fine and extrafine formulations delivered from dry powder inhalers, metered dose inhalers, and soft mist inhalers (SMI) (FIG. 11).

The ratio of TLD (i.e., lower respiratory tract) deposition to extrathoracic (i.e., upper respiratory tract) deposition is 13.3 for Example 11 (93.0% TLD/7.0% URT). This is 5-fold higher than all marketed ICS products, including budesonide administered with the high efficiency Respimat® SMI. Lung targeting is improved 55-fold relative to the top-selling Advair® Diskus®.

The improved lung targeting noted with CPI is expected to reduce local adverse events in the URT including throat irritation, dysphonia, and opportunistic infections (e.g., candidiasis and descending pneumonia). For ICS with oral bioavailability, the reduced throat deposition will reduce systemic exposure and resulting systemic adverse events including growth delay, renal insufficiency, and effects on bone mineral accretion. The improved lung targeting may also enable reductions in nominal dose, not only due to the improved targeting, but also because of the reduced variability in TLD.

### Example 11

### Deposition of ICS Formulations in the Idealized Child Throat (ICT)

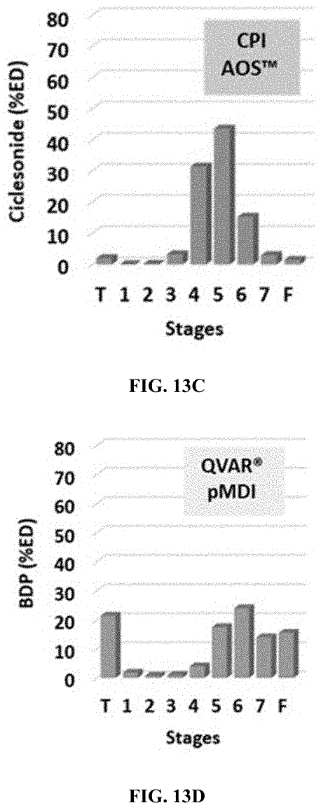

The deposition of ICS in the device, ICT, and filter (representing the TLD) for three ICS formulations, including CPI (Example 11) is shown in FIG. 12. Strong in vitro-in vivo correlations were established in the ICT model for Pulmicort® Turbuhaler® and QVAR® by Ruzycki et al. (Pharm Res. 2014; 31:1525-1531).

Relative to these two ICS formulations, CPI had significantly reduced device and URT deposition. When expressed as a percentage of the emitted dose (ED), deposition in the ICT was 69.0% for Pulmicort, 39.2% for QVAR, and 13.5% for CPI. TLD values increased from 31.0% for Pulmicort to 60.8% for QVAR to 86.5% for CPI. The ratio of TLD/ICT deposition was 0.45 for Pulmicort, 1.55 for QVAR, and 6.41 for CPI. Thus, CPI enables significant improvements in lung targeting in a pediatric throat model compared to marketed DPI and extrafine pMDI formulations.

### Example 12

### Comparison of Aerodynamic Particle Size Distributions (aPSD) of ICS Formulations

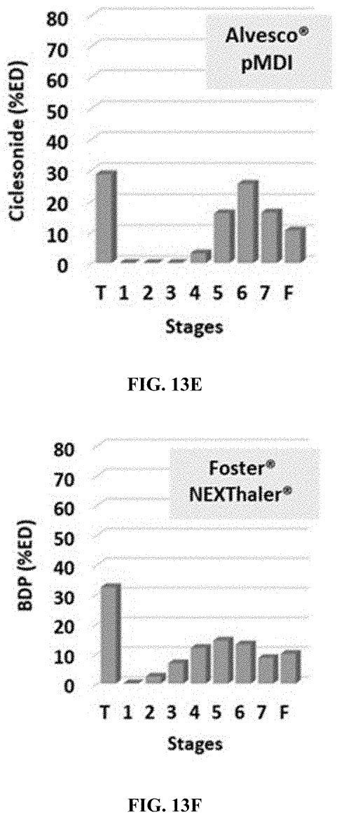

The aPSDs of various ICS formulations are detailed in FIGS. 13A-13F. FIGS. 13A-13C show two leading lactose blend formulations of mometasone furoate (Asmanex® Twisthaler®) and fluticasone propionate (Flovent® Diskus®), and the CPI formulation of the present disclosure (Example 11). For CPI, only 2.5% of the emitted dose is deposited in the throat/induction port (T) and Stages 1 and 2 of the Next Generation Impactor. The bulk of the deposition occurs on stages 4 to 6 in the impactor, with small amounts of deposition on stage 7 and filter. In contrast, the Asmanex and Flovent DPI formulations deposit most of their dose in the throat and pre-separator. Overall, it appears that for CPI, drug that bypassed the throat is instead deposited in the lungs, with a significant proportion in the small airways.

FIGS. 13D-13F show the aPSD profiles for ‘extrafine’ solution pMDI and DPI formulations. Throat deposition is increased by more than 10-fold for these formulations relative to CPI. As well, deposition on stage 7 and filter is also increased for these ‘extrafine’ formulations.

### Example 13

### Targeted Delivery to the Airways

Table 12 compares stage grouping metrics for various ICS formulations based on their NGI™ stage distributions. CPI is clearly unique in its stage distribution. Relative to other extrafine formulations, CPI has limited deposition on S7-F, thereby decreasing the potential for alveolar delivery and particle exhalation. This is reflected in much higher values of ξ. While the values of ξ are also high for fine-particle DPI formulations, this is more of a reflection that these products are likely to deposit very little of their emitted dose in the peripheral regions of the lungs.

TABLE 12

| | Asmanex | Flovent | Alvesco | QVAR | Foster | |
|---|---|---|---|---|---|---|
| | Twisthaler | Diskus | pMDI | pMDI | NextHALER | CPI |
| Aerosol Metric | (% ED) | (% ED) | (% ED) | (% ED) | (% ED) | (% ED) |
| ξ (S3-S6/S7-F) | 10.35 | 194 | 1.67 | 1.58 | 2.49 | 22.2 |
| ϑ (S5-S6/S3-S4) | 0.31 | 0.36 | 13.42 | 8.30 | 1.47 | 1.82 |
| | Fine | Fine | Extrafine | Extrafine | Extrafine | Extrafine |

Improved targeting to the small airways, as reflected by increases in ϑ, is also observed for CPI relative to the fine particle DPI formulations by approximately 6-fold. The high values of ϑ observed for extrafine solution pMDIs is the result of very little deposition on stages 3-4. Deposition on these stages is deemed important for effective delivery to the large airways.

Hence, CPI seemingly balances the desire to largely bypass deposition in the URT while also effectively delivering drug to both the large and small airways, yet limiting alveolar deposition and particle exhalation.

### Example 14

### Leucine Carrier Particles Prepared From Organic Co-Solvent Feedstock

Table 13 provides the particle properties of leucine carrier particles prepared from organic co-solvent feedstock. Leucine carrier particles were prepared from a feedstock that included a small amount (0 to 15% w/w) of organic co-solvent. Examples 20-24 provide five leucine powder batches prepared from solutions with 1% solids and Examples 25-27 were prepared from saturated solutions that were filtered (using a 0.22 μm membrane) prior to spray drying. Spray drying was conducted as described in Example 1. The examples demonstrate that alcohols such as ethanol and 2-propanol decrease the surface tension of the feedstock and reduce the atomized droplet size. Additionally, depending on the relative evaporation rates of the alcohol and water, the addition of alcohol can result in earlier particle formation due to the reduction in the solubility of leucine in the mixed solvent.

Table 13 shows that Examples 20-26, each comprising a feedstock including ethanol, achieved a tapped density that ranged from 0.034 g/cm³ to 0.050 g/cm³. While the solids content did not affect the tapped density, the use of ethanol had a modest effect on tapped density, with the lowest densities measured for the examples spray-dried from 7.5% ethanol (1% solids) and 5% ethanol (1.8% solids). Example 27 was spray dried using 2-propanol as a cosolvent and was more dense (0.057 g/cm³) than any of the dry powder produced from a feedstock including ethanol (Examples 20-26).

The primary particle size (x₅₀) of most dry powders in the examples was approximately 2.0 μm, with the single exception being Example 24 which spray-dried from a solution at the highest solids content. Examples 20-27 all achieved Dₐ values less than 0.5 μm. Comparison of the Dₐ values of Example 20 and Example 21 with the Dₐ values of Example 28 (prepared without ethanol) indicates that there is an advantage in using ethanol to enable a lower Dₐ value at the same solids content.

TABLE 13

| | | Organic | Solids | Tapped | | |
|---|---|---|---|---|---|---|
| | Organic | cosolvent | content | density | x₅₀ | Dₐ |
| | cosolvent | (% w/w) | (% w/v) | (g/cm³) | (μm) | (μm) |
| Ex. 20 | Ethanol | 5 | 1 | 0.043 | 1.86 | 0.38 |
| Ex. 21 | Ethanol | 7.5 | 1 | 0.036 | 2.00 | 0.38 |
| Ex. 22 | Ethanol | 10 | 1 | 0.045 | 1.98 | 0.42 |
| Ex. 23 | Ethanol | 15 | 1 | 0.044 | 1.93 | 0.41 |
| Ex. 24 | Ethanol | 5 | 1.8 | 0.034 | 2.36 | 0.43 |
| Ex. 25 | Ethanol | 10 | 1.5 | 0.050 | 1.93 | 0.43 |
| Ex. 26 | Ethanol | 15 | 1.2 | 0.045 | 1.91 | 0.41 |
| Ex. 27 | 2-propanol | 5 | 1 | 0.057 | 1.89 | 0.45 |
| Ex. 28 | — | 0 | 1 | 0.040 | 2.08 | 0.42 |

### Example 15

### Preparing of Ciclesonide/Leucine Blends Using Different Drying Techniques

Table 14 provides the particle properties of 1% (w/w) ciclesonide/leucine blends using different drying processes. A single ciclesonide/leucine feedstock was prepared as described in Example 5 and then divided into three aliquots for further processing using spray drying, vacuum drying, and freeze drying. The feedstock was formulated to include 1% w/w ciclesonide. Spray drying was conducted as described in Example 2.

Vacuum drying was conducted at ambient temperature using a VWR vacuum oven and a Welch DryFast Ultra diaphragm vacuum pump (ultimate pressure=270 Pa). At ambient temperature, this pressure does not result in boiling of PFOB. Approximately 45 g of feedstock was poured in a 7 mm layer in a 250 mL glass jar. Using this approach, the evaporation rate was approximately 30 g/h.

Freeze-drying was conducted using a custom-built apparatus that consisted of an Edwards 2 E2M2 rotary vane vacuum pump, two vacuum chambers, and an Accutools BluVac+ digital vacuum gauge. The first vacuum chamber served as a condenser and was cooled with dry ice (-78° C.). The second chamber contained the sample to be dried and was located distal to the vacuum pump. Approximately 46 g of feedstock was poured into a 7 mm layer in a 250 mL glass jar and placed in a laboratory freezer for approximately 2 hours at −13° C. The frozen ciclesonide/leucine blend suspended in PFOB was then placed inside the sample chamber which was cooled with a mixture of ice and calcium chloride (approximately −20° C.). Drying was conducted by application of low vacuum (74 Pa) for 16 hours.

As shown in Table 14, the yield of Examples 30 and 31 is effectively 100% for these processes given that vacuum drying and freeze drying use a confined sample. For spray drying, the yield of Example 31 was approximately 56% due to the collection efficiency of fine particles during drying as well as a small amount of residual feedstock in the container after spray drying.

While spray-drying and freeze-drying resulted in loose, flowable powders, vacuum drying produced a dense, cracked powder cake. After vacuum drying, the friable cake was comminuted by stirring with a magnetic stir bar at low speed (200 RPM) for about two minutes.

The tapped density of the freeze-dried sample of Example 29 was the lowest, followed by the spray-dried sample of Example 31, and then the (comminuted) vacuum-dried sample of Example 30 being significantly more dense

TABLE 14

| | | Process | Tapped | | |
|---|---|---|---|---|---|
| | | yield | density | x₅₀ | Dₐ |
| | Process | (%) | (g/cm³) | (μm) | (μm) |
| Ex. 29 | Freeze-dried | 100% | 0.050 | 2.34 | 0.52 |
| Ex. 30 | Vacuum-dried | 100% | 0.134 | 2.29 | 0.83 |
| Ex. 31 | Spray-dried | 56% | 0.063 | 1.92 | 0.49 |

Additionally, Table 14 shows that the primary particle size (x₅₀) of freeze-dried and vacuum-dried powders was greater than that of the spray-dried powder. Owing to its greater density and primary particle size, the vacuum-dried powder had the largest Dₐ value. The freeze-dried and spray-dried powders had comparable Dₐ values.

The foregoing description of certain aspects and features, including illustrated embodiments, has been presented only for the purpose of illustration and description and is not intended to be exhaustive or to limit the disclosure to the precise forms disclosed. Numerous modifications, adaptations, and uses thereof will be apparent to those skilled in the art without departing from the scope of the disclosure. Certain features that are described in this specification in the context of separate embodiments can also be implemented in combination in a single implementation. Conversely, various features that are described in the context of a single implementation can also be implemented in multiple ways separately or in any suitable sub-combination. Moreover, although features may be described above as acting in certain combinations, one or more features from a combination can in some cases be excised from the combination, and the combination may be directed to a sub-combination or variation of a sub-combination. Thus, particular embodiments have been described. Other embodiments are within the scope of the disclosure.

The entire disclosure of each reference, United States patent, U.S. patent application, and international patent application mentioned in this patent specification is fully incorporated by reference herein for all purposes.

What is claimed:

1. A carrier-based dry powder formulation comprising a plurality of drug particles adhered to extrafine leucine carrier particles forming particle agglomerates having a mass median impaction parameter (MMIP) value between 50 and 500 μm² L min⁻¹.

2. The formulation of claim 1, wherein a median aerodynamic diameter of the extrafine leucine carrier particles (Dₐ) is less than 1000 nm.

3. The formulation of claim 1, wherein a median aerodynamic diameter of the extrafine leucine carrier particles (Dₐ) is about 300 to 700 nm.

4. The formulation of claim 1, wherein the extrafine leucine carrier particles have a crystallinity greater than 90%.

5. The formulation of claim 1, wherein the drug particles have a mass median diameter less than 3 μm.

6. The formulation of claim 1, wherein the drug particles have a mass median diameter of about 20 nm to 500 nm.

7. The formulation of claim 1, wherein the drug particles have a crystallinity greater than 90%.

8. The formulation of claim 1, wherein the drug particles have an amorphous content greater than 90%.

9. The formulation of claim 1, wherein the drug particles have a total lung dose in Alberta Idealized Throat of greater than 90% of an emitted dose.

10. The formulation of claim 1, wherein the drug particles comprise one or more corticosteroids, one or more bronchodilators, or any combinations thereof.

11. The formulation of claim 1, wherein greater than 70% of an emitted dose of the carrier-based dry powder formulation is delivered to at least one of stages 4, 5, and 6 of a NEXT GENERATION IMPACTOR™ (NGI).

12. A carrier-based dry powder formulation comprising a plurality of drug particles adhered to fine leucine carrier particles forming particle agglomerates having a mass median impaction parameter (MMIP) value between 500 and 2500 μm² L min⁻¹.

13. The formulation of claim 12, wherein a median aerodynamic diameter of the fine leucine carrier particles (Dₐ) is between 1 μm and 5 μm.

14. The formulation of claim 12, wherein a crystallinity of the fine leucine carrier particles is greater than 90%.

15. The formulation of claim 12, wherein the drug particles have a mass median diameter less than 3 μm.

16. The formulation of claim 12, wherein the drug particles have a crystallinity greater than 90%.

17. The formulation of claim 12, wherein the drug particles have an amorphous content greater than 90%.

18. The formulation of claim 12, wherein the drug particles comprise one or more corticosteroids, one or more bronchodilators, or any combinations thereof.

19. The formulation of claim 12, wherein the drug particles have a total lung dose in Alberta Idealized Throat of greater than 70% of an emitted dose.

20. The formulation of claim 12, wherein greater than 70% of an emitted of the carrier-based dry powder formulation is delivered to at least one of stages 3, 4, and 5of a NEXT GENERATION IMPACTOR™ (NGI).

21. A method of preparing a carrier-based dry powder formulation, the method comprising:
preparing an aqueous solution comprising leucine and a first solvent;
drying the aqueous solution to produce extrafine leucine carrier particles comprising a median aerodynamic diameter (Dₐ) less than 1000 nm;
adding a non-solvent to the extrafine leucine carrier particles to form a suspension;
preparing a drug solution comprising a drug and a second solvent that is miscible with the non-solvent;
adding the drug solution to the suspension of extrafine leucine carrier particles in the non-solvent while mixing to precipitate the drug particles thereby forming a co-suspension of drug particles and extrafine leucine carrier particles in the non-solvent; and
removing the non-solvent to form a dry powder comprising an adhesive mixture of drug particles adhered to the extrafine leucine carrier particles, wherein the adhesive mixture has a mass median impaction parameter (MMIP) value between 50 and 500 μm² L min⁻¹.

22. The method of claim 21, wherein the first solvent is water, ethanol, or a combination thereof.

23. The method of claim 21, wherein a solids content of the leucine in the first solvent is from 0.4% w/w and 1.8% w/w.

24. The method of claim 21, wherein drying the aqueous solution to produce the extrafine leucine carrier particles is performed by spray drying.

25. The method of claim 21, wherein non-solvent is a perfluorinated liquid or a fluorocarbon-hydrocarbon diblock.

26. The method of claim 25, wherein the non-solvent is perfluorooctyl bromide, perfluorodecalin, perfluorooctyl ethane, perfluorohexyl butane, or perfluorohexyl decane.

27. The method of claim 21, wherein the drug particles have a crystallinity greater than 90%.

28. The method of claim 21, wherein the drug solution is added dropwise to the suspension.

29. The method of claim 21, further comprising removing the non-solvent by spray drying the co-suspension to produce a dry powder.

30. The method of claim 21, further comprising removing the non-solvent by lyophilizing the co-suspension to produce a dry powder.

31. The method of claim 21, wherein the extrafine leucine carrier particles have a (Dₐ) from 300 nm to 700 nm and a tapped density from 0.01 g/cm³ to 0.30 g/cm³.

32. The method of claim 21, wherein the second solvent comprises 2-propanol.

33. The method of claim 21, wherein a blend uniformity of the drug solution in the co-suspension has a standard deviation less than 2%.

34. A method of treating a disease in a subject, the method comprising administering to a subject in need thereof an effective amount of a carrier-based dry powder formulation of claim 1 or claim 12, wherein the carrier-based dry powder formulation is administered to the subject via inhalation.

35. The method of claim 34, wherein the carrier-based dry powder formulation is administered as an aerosol.

36. The method of claim 34, wherein the carrier-based dry powder formulation is administered using a metered dose inhaler, a dry powder inhaler, a single dose inhaler, or a multi-unit dose inhaler.

37. The method of claim 34, wherein the carrier-based dry powder formulation is administered by
providing an inhaler comprising a dispersion chamber having an inlet and an outlet, the dispersion chamber containing an actuator that is configured to oscillate along a longitudinal axis of the dispersion chamber; and
inducing air flow through the outlet channel to cause air and the carrier-based dry powder formulation to enter into the dispersion chamber from the inlet, and to cause the actuator to oscillate within the dispersion chamber to assist in dispersing the carrier-based dry powder formulation from the outlet for delivery to the subject through the outlet.

38. The method of claim 34, wherein greater than 70% of the carrier-based dry powder formulation administered to the subject is delivered to the lungs of the subject.

39. The method of claim 34, wherein a portion of the carrier-based dry powder formulation is delivered to peripheral regions of the lungs of the subject.

40. The method of claim 34, wherein the disease is a pulmonary disease.

41. The method of claim 34, wherein the disease is at least one of a chronic obstructive pulmonary disease, asthma, interstitial lung disease, an airway infection, a connective tissues disease, an inflammatory bowel disease, bone marrow or lung transplantation, an immune deficiency, diffuse panbronchiolitis, bronchiolitis, or mineral dust airway disease.
